(19) United States
(12) Patent Application Publication
(10) Pub. No.: US 2026/0157111 A1
(43) Pub. Date: Jun. 4, 2026

(54) THERMAL EVAPORATION OF NAPHTHALENE DIIMIDE ELECTRON TRANSPORT LAYERS FOR OPTOELECTRONIC APPLICATIONS

(71) Applicant: Georgia Tech Research Corporation, Atlanta, GA (US)

(72) Inventors: Juan-Pablo Correa-Baena, Altanta, GA (US); John R. Reynolds, Dunwoody, GA (US); Jack Lawton, Atlanta, GA (US); Justine Sina Wagner, Converse, TX (US); Carlo Andrea Riccardo Perini, Atlanta, GA (US)

(21) Appl. No.: 19/404,499

(22) Filed: Dec. 1, 2025

Related U.S. Application Data
(60) Provisional application No. 63/726,878, filed on Dec. 2, 2024.

(51) Int. Cl. H10K 85/60; C07D 471/06; C07F 9/6561; C09D 5/24; H10K 30/85; H10K 71/12

(52) U.S. Cl. CPC H10K 85/6572; C07D 471/06; C07F 9/6561; C09D 5/24; H10K 71/12; H10K 30/85

(57) ABSTRACT

Provided herein are naphthalene diimide (NDI) compounds and their use in thin films, methods of making thin films, photovoltaic devices comprising NDI thin films, and methods of making photovoltaic devices comprising NDI thin films. Additionally contemplated are organic light-emitting diodes comprising NDI thin films, photodetectors comprising NDI thin films, and organic field-effect transistors (OFETs) comprising NDI thin films.

### RELATED APPLICATIONS

This application claims the benefit of priority to U.S. Provisional Patent Application No. 63/726,878, filed Dec. 2, 2024, the contents of which are incorporated herein by reference in their entirety.

### BACKGROUND

Perovskite solar cells (PSCs) have exhibited a rapid in power conversion efficiency (PCE) since their inception over a decade ago. The excitement around PSCs has been bolstered by their rapid advancement and the promise of low-cost and scalable device fabrication. The electron transport layer (ETL) is an essential component of a high-performance PSC that facilitates the extraction and transport of electrons from the perovskite layer to the electrode while simultaneously blocking holes. Traditionally, inorganic ETLs such as TiO₂ and SnO₂ have been used, but recent focus has shifted towards organic ETLs due to their low processing temperatures, chemical adaptability and compatibility with thermal evaporation. However, there are few reports on the development of new organic ETLs that can be processed via both solution and evaporation. The most commonly used organic ETLs are fullerene-based materials, such as C₆₀ and [6,6]-phenyl-C₆₁-butyric acid methyl ester (PCBM). Despite advantageous charge transport properties, these materials suffer from poor frontier orbital tunability, high synthetic costs and poor ambient stability which has motivated the development of non-fullerene based organic-ETLs. Naphthalene diimide (NDI) and its derivatives provide an alternative route to achieving high efficiency PSCs that incorporate organic ETLs. NDIs have been extensively used in organic electronics owing to their electron affinity, high electron mobilities, tunable optical absorption profiles, and exceptional thermal stability. A major hurdle, which has limited the widespread incorporation of NDI research into conventional PSC architecture, is the insolubility of NDI-H in common process solvents. In view of the foregoing, there is an unmet need for NDIs with improved solubility, and for new methods of fabricating NDI-H PSCs.

### SUMMARY OF THE INVENTION

In certain aspects, the present disclosure provides compounds of Formula (I) or Formula (II):

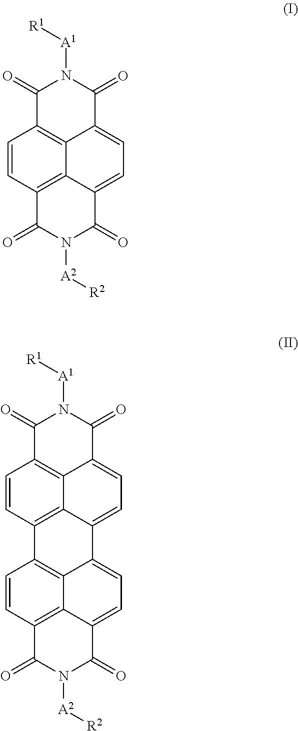

wherein:
A¹ is alkylene or arylene;
A² is selected from the group consisting of alkylene, alkoxylene, or arylene;
R¹ is selected from the group consisting of phosphate, phosphonate, -alkylene-phosphate, -alkylene-phosphonate, and halo; and
R² is selected from the group consisting of phosphate, phosphonate, -alkylene-phosphate, -alkylene-phosphonate, halo, and alkyl.

In further aspects, the present disclosure provides thin films comprising a compound of this disclosure.

In yet further aspects, the present disclosure provides methods of making a thin film, the methods comprising:
combining a compound of this disclosure with a solvent, thereby forming a first mixture;
contacting a substrate with the first mixture, thereby forming a treated substrate; and heating the treated substrate, thereby forming the thin film.

In still further aspects, the present disclosure provides photovoltaics device comprising:
a substrate;
an electron transport layer comprising a compound of this disclosure;
an active layer;
an interface layer;
a hole transport layer; and
a metal layer.

In certain aspects, the present disclosure provides methods of making a photovoltaic device, the method comprising:
contacting a substrate with a first solution comprising a compound of this disclosure, thereby forming an electron transport layer disposed along at least one surface of the substrate;
contacting the electron transport layer with a second solution comprising an active layer precursor, thereby forming an active layer disposed along at least one surface of the electron transport layer;
contacting the active layer with a third solution comprising an interface compound, thereby forming an interface layer disposed along at least one surface of the electron transport layer;
contacting the interface layer with a fourth solution comprising a hole transport compound, thereby forming a hole transport layer disposed along at least one surface of the interface layer;
contacting the hole transport layer with a metal, thereby forming a metal layer disposed along at least one surface of the hole transport layer;
thereby forming the photovoltaic device.

In further aspects, the present disclosure provides organic light-emitting diodes (OLEDs) comprising a thin film of this disclosure.

In yet further aspects, the present disclosure provides photodetectors comprising a thin film of this disclosure.

In still further aspects, the present disclosure provides organic field-effect transistors (OFETs) comprising a compound of this disclosure.

### BRIEF DESCRIPTION OF THE DRAWINGS

FIG. 1A shows the molecular structures of the 7 NDI derivatives and 1 PDI.

FIG. 1B depicts a thermogravimetric analysis plot of NDI-(EtPA)₂ and NDI-(PhBr)₂, measured at a ramp rate of 15° C./min under a N₂ atmosphere.

FIG. 2A depicts the FTIR spectrum in the fingerprint region (1900 cm⁻¹-500 cm⁻¹) for thin films of NDI-(PhBr)₂ (top) and NDI-(EtPA)₂ (bottom) deposited via evaporation and solution.

FIG. 2B depicts the NDI core with bonds exhibiting identifiable vibrational frequencies highlighted.

FIG. 2C depicts N is XPS spectra for NDI-(PhBr)₂ and NDI-(EtPA)₂. The spheres represent the recorded data, while the lines represents the fitted envelope.

FIG. 2D depicts P 2p XPS spectra for NDI-(EtPA)₂ processed via solution (light grey) and evaporation (black).

FIG. 2E depicts optical absorption spectra for NDI-(PhBr)₂ and NDI-(EtPA)₂ thin films deposited via thermal evaporation.

FIG. 3A depicts FTIR spectra of NDI-(PhBr)₂ and NDI-(EtPA)₂ before and after stressing at 90° C. for 12 hours in air (with 60% relative humidity) conditions under white light (1 sun equivalent).

FIG. 3B depicts DSC thermograms of both NDI-(PhBr)₂ (top graph) and NDI-(EtPA)₂ (bottom graph).

FIG. 4A depicts 2D GIWAXS scattering pattern for NDI-(EtPA)₂ before (left) and after (right) solvent washing.

FIG. 4B depicts 2D GIWAXS scattering pattern for NDI-(PhBr)₂ before (left) and after (right) solvent washing.

FIG. 4C depicts N is XPS of NDI-(EtPA)₂ and NDI-(PhBr)₂ before and after solvent washing.

FIG. 4D depicts N is XPS of NDI-(EtPA)₂ after washing. The spheres represent the recorded data, while the lines represents the fitted envelope.

FIG. 5A depicts reverse J-V scan for both NDI-(EtPA)₂ and NDI-(PhBr)₂.

FIG. 5B depicts a PCE box plot for NDI-(EtPA)₂, NDI-(PhBr)₂ and reference devices. The solid points represent individual data points, the horizontal bar represents the median value of a distribution while the box signifies the interquartile range.

FIG. 5C depicts MPP tracking of NDI-(EtPA)₂, NDI-(PhBr)₂, the TiO₂ reference devices, and devices without an ETL over a 200-hour period at a temperature of 65° C. The distribution shows the standard deviation associated with a particular time point.

FIG. 6 depicts DSC Thermograms for NDI-(PhBr)(BzPA), NDI-(BzPA)(Hex) and NDI-(PhBr)(Hex).

FIG. 7 depicts a schematic of the evaporation chamber used throughout the disclosure.

FIG. 8A depicts evaporation data recorded during thermal evaporation of NDI-(EtPA)₂ (Top): Pressure, (Middle): Deposition rate, (Bottom): Temperature.

FIG. 8B depicts evaporation data recorded during thermal evaporation of NDI-(PhBr)₂ (Top): Pressure, (Middle): Deposition rate, (Bottom): Temperature.

FIG. 9 depicts residual powder remaining in crucible after the deposition of NDI-(EtPA)₂.

FIG. 10A depicts an overlay of the solution processed and thermally evaporated thin films of NDI-(EtPA)₂.

FIG. 10B depicts an overlay of the solution processed and thermally evaporated thin films of NDI-(PhBr)₂.

FIG. 11 depicts N is XPS spectra for NDI-(EtPA)₂ processed via solution (light grey) and evaporation (black).

FIG. 12A depicts a Tauc plot of NDI-(EtPA)₂.

FIG. 12B depicts a Tauc plot of NDI-(PhBr)₂.

FIG. 13 depicts a schematic of the solvent wash process undertaken to simulate the effects of solvents during perovskite deposition on top of the NDI films.

FIG. 14A depicts the contact angle of NDI-(EtPA)₂ with H₂O.

FIG. 14B depicts the contact angle of NDI-(PhBr)₂ with H₂O.

FIG. 14C depicts the contact angle of FTO with H₂O.

FIG. 15 depicts the P 2p XPS spectrum of an NDI-(EtPA)₂ film after the solvent wash process.

FIG. 16 depicts the P 2p XPS spectrum of bare FTO.

FIG. 17 depicts a schematic of the perovskite solar cell architecture used in this disclosure.

FIG. 18A is a box plot depicting the distribution in V_{OC} for NDI-(EtPA)₂, NDI-(PhBr)₂, and a TiO₂ reference. The solid points represent individual data points, while the hollow points represent the mean of a given distribution. The horizontal bar represents the median value of a distribution while the box signifies the interquartile range.

FIG. 18B is a box plot depicting the distribution in J_{SC} for NDI-(EtPA)₂, NDI-(PhBr)₂, and a TiO₂ reference. The solid points represent individual data points, while the hollow points represent the mean of a given distribution. The horizontal bar represents the median value of a distribution while the box signifies the interquartile range.

FIG. 18C is a box plot depicting the distribution in Fill Factor (FF) for NDI-(EtPA)₂, NDI-(PhBr)₂, and a TiO₂ reference. The solid points represent individual data points, while the hollow points represent the mean of a given distribution. The horizontal bar represents the median value of a distribution while the box signifies the interquartile range.

FIG. 19 depicts a stabilized PCE of NDI-(EtPA)₂, NDI-(PhBr)₂, the TiO₂ reference devices, and devices without an ETL.

FIG. 20A shows the change in contact angle for NDI-dPA (also referred to as NDI-(EtPA)₂) and NDI-dBr (also referred to as NDI-(PhBr)₂) before and after the solvent wash procedure.

FIG. 20B shows the N1 XPS of NDI-dPA and NDI-dBr before (solid) and after (dashed) solvent wash.

FIG. 21 shows a TGA analysis of NDI-3, 4, 5, and 6.

FIG. 22 shows variations in deposition rate, pressure and temperature during deposition of NDI-1.

FIG. 23 shows variations in deposition rate, pressure and temperature during deposition of NDI-2.

FIG. 24 shows variations in deposition rate, pressure and temperature during deposition of NDI-3.

FIG. 25 shows variations in deposition rate, pressure and temperature during deposition of NDI-4.

FIG. 26 shows variations in deposition rate, pressure and temperature during deposition of NDI-5.

FIG. 27 shows variations in deposition rate, pressure and temperature during deposition of NDI-6.

FIG. 28 shows an FTIR spectrum of NDI-3 thin film deposited via evaporation.

FIG. 29 shows an FTIR spectrum of material remaining in crucible after deposition of NDI-4.

FIG. 30 shows an FTIR spectrum of material remaining in crucible after deposition of NDI-5.

FIG. 31 shows an FTIR spectrum of material remaining in crucible after deposition of NDI-6.

FIG. 32 shows an FTIR comparison of precursor powder of NDI-4 and remaining material in crucible after deposition.

FIG. 33 shows UV-Vis absorption spectra for NDI-3,4,5 and 6.

FIG. 34 shows Tauc plots for NDI-1,2,3,4,5 and 6.

FIG. 35 shows the complete UPS spectrum for NDI-dPA.

FIG. 36 shows the complete UPS spectrum for NDI-dBr.

FIG. 37 shows the HOMO onset for NDI-1 and NDI-2.

FIG. 38 shows the HOMO onset for NDI-1 and NDI-2.

FIG. 39 shows FTIR spectra for NDI-3 before and after stressing in an ambient atmosphere at 90° C. for 12 hours.

FIG. 40 shows FTIR spectra for NDI-4 before and after stressing in an ambient atmosphere at 90° C. for 12 hours.

FIG. 41 shows FTIR spectra for NDI-5 before and after stressing in an ambient atmosphere at 90° C. for 12 hours.

FIG. 42 shows FTIR spectra for NDI-6 before and after stressing in an ambient atmosphere at 90° C. for 12 hours.

FIG. 43 shows H₂O contact angle images for NDI-1,2,3,4,5 and 6 before and after the solvent wash.

FIG. 44 shows dark JV scans for NDI-1 and NDI-2.

### DETAILED DESCRIPTION OF THE INVENTION

In certain aspects, the present disclosure provides compounds of Formula (I) or Formula (II):

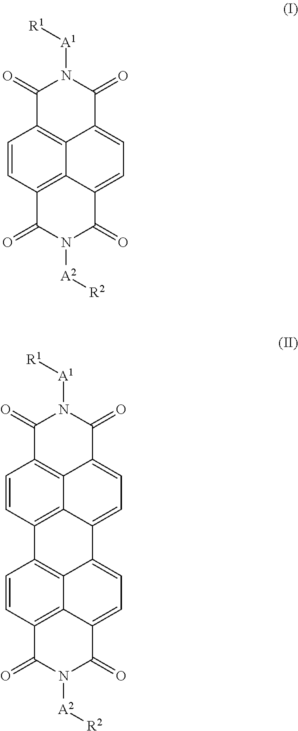

wherein:
A¹ is alkylene or arylene;
A² is selected from the group consisting of alkylene, alkoxylene, or arylene;
R¹ is selected from the group consisting of phosphate, phosphonate, -alkylene-phosphate, -alkylene-phosphonate, and halo; and
R² is selected from the group consisting of phosphate, phosphonate, -alkylene-phosphate, -alkylene-phosphonate, halo, and alkyl.

In certain embodiments, A¹ is alkylene. In further embodiments, A¹ is methylene. In yet further embodiments, A¹ is ethylene. In still further embodiments, A¹ is arylene. In certain embodiments, A¹ is phenylene.

In further embodiments, A² is alkylene. In yet further embodiments, A² is methylene. In still further embodiments, A² is ethylene. In certain embodiments, A² is alkoxylene. In further embodiments, A² is

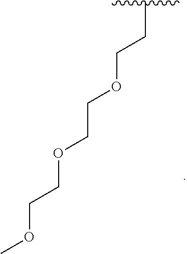 [SMILES: *CCOCCOCCOC]

In yet further embodiments, A² is arylene. In still further embodiments, A² is phenylene.

In certain embodiments, R¹ is phosphate. In further embodiments, R¹ is phosphonate. In yet further embodiments, R¹ is -alkylene-phosphate. In still further embodiments, R¹ is -alkylene-phosphonate. In certain embodiments, R¹ is -methylene-phosphonate. In further embodiments, R¹ is -ethylene-phosphonate. In yet further embodiments, R¹ is halo. In still further embodiments, R¹ is bromo.

In certain embodiments, R² is phosphate. In further embodiments, R² is phosphonate. In yet further embodiments, R² is -alkylene-phosphate. In still further embodiments, R² is -alkylene-phosphonate. In certain embodiments, R² is -methylene-phosphonate. In further embodiments, R² is -ethylene-phosphonate. In yet further embodiments, R² is halo. In still further embodiments, R² is bromo. In certain embodiments, R² is alkyl. In further embodiments, R² is hexyl.

In yet further embodiments, the compound is selected from the group consisting of:

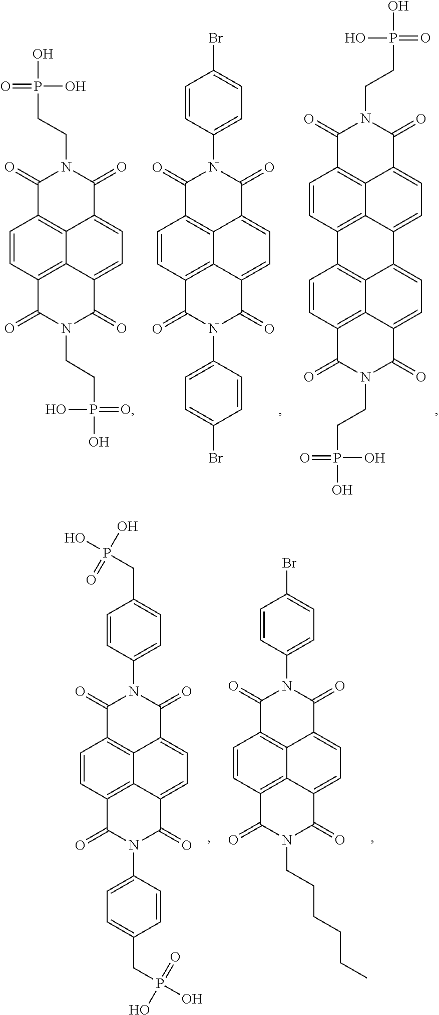

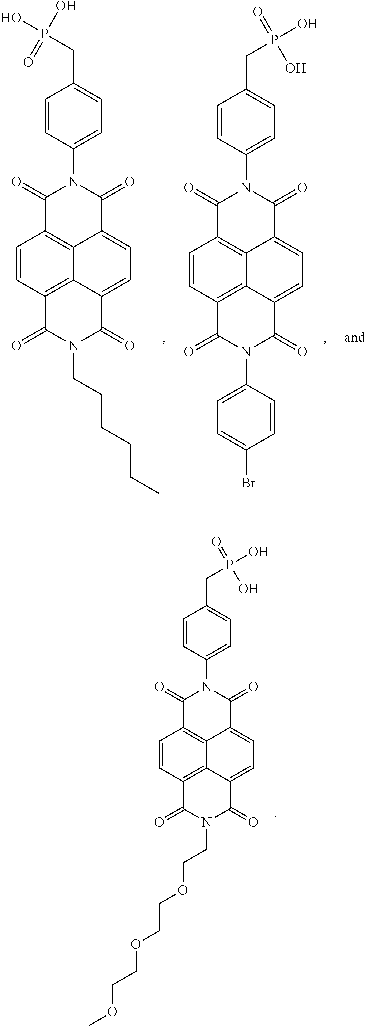 [SMILES: CCCCCCN1C(=O)c2ccc3c4c(ccc(c24)C1=O)C(=O)N(c1ccc(CP(=O)(O)O)cc1)C3=O ; O=C1c2ccc3c4c(ccc(c24)C(=O)N1c1ccc(Br)cc1)C(=O)N(c1ccc(CP(=O)(O)O)cc1)C3=O ; COCCOCCOCCN1C(=O)c2ccc3c4c(ccc(c24)C1=O)C(=O)N(c1ccc(CP(=O)(O)O)cc1)C3=O]

In further aspects, the present disclosure provides thin films comprising a compound of the present disclosure.

In certain embodiments, the thin film is produced by thermal evaporation. In further embodiments, the thin film is produced by solution coating. In yet further embodiments, the solution coating is spin coating. In still further embodiments, the solution coating is slot die coating. In certain embodiments, the solution coating is blade coating. In further embodiments, the solution coating is inkjet printing.

In yet further embodiments, the thin film has an FTIR absorbance spectrum comprising a first local maximum comprising two peaks from about 1600-1800 cm⁻¹. In still further embodiments, the thin film has an FTIR absorbance spectrum comprising a first local maximum comprising two peaks from about 1625-1750 cm⁻¹. In certain embodiments, the thin film has an FTIR absorbance spectrum comprising a second local maximum comprising one peak from about 1500-1600 cm⁻¹. In further embodiments, the thin film has an FTIR absorbance spectrum comprising a second local maximum at about 1580 cm⁻¹. In yet further embodiments, the thin film has an FTIR absorbance spectrum comprising a third local maximum comprising one peak from about 1200-1300 cm⁻¹. In still further embodiments, the thin film has an FTIR absorbance spectrum comprising a third local maximum at about 1250 cm⁻¹.

In certain embodiments, the thin film has a N is X-ray photoelectron (XPS) spectrum comprising a first global maximum from about 380-420 eV. In further embodiments, the thin film has a N is X-ray photoelectron (XPS) spectrum comprising a first global maximum from about 398-402 eV. In yet further embodiments, the thin film has a P 2p XPS spectrum comprising a second global maximum from about 120-150 eV. In still further embodiments, the thin film has a P 2p XPS spectrum comprising a second global maximum from about 132-136 eV.

In certain embodiments, the thin film has a UV-Vis absorbance spectrum comprising a third global maximum from about 300-450 nm. In further embodiments, the thin film has a UV-Vis absorbance spectrum comprising a third global maximum from about 375-425 nm. In yet further embodiments, the thin film has a UV-Vis absorbance spectrum comprising a third global maximum at about 393 nm.

In still further embodiments, the thin film has an optical band gap energy from about 2.5-3.5 eV. In certain embodiments, the thin film has an optical band gap energy from about 2.9-3.1 eV. In further embodiments, the thin film has an optical band gap energy of about 2.95 eV or about 2.96 eV.

In yet further embodiments, the thin film is substantially thermally stable up to about 100° C. over a period of about 24 hours. In still further embodiments, the thin film is substantially thermally stable at about 90° C. over a period of 12 hours. In certain embodiments, the thin film is substantially photochemically stable to white light. In further embodiments, the thin film is substantially photochemically stable to ultraviolet light. In yet further embodiments, the thin film is substantially inert to solvent. In still further embodiments, the solvent is dimethylformamide (DMF) or dimethyl sulfoxide (DMSO).

In certain embodiments, the thin film has an electron mobility of about 10⁻⁵-10¹ cm²/(V·s).

In yet further aspects, the present disclosure provides methods of making a thin film, the method comprising:
combining a compound of the present disclosure with a solvent, thereby forming a first mixture;
contacting a substrate with the first mixture, thereby forming a treated substrate; and heating the treated substrate, thereby forming the thin film.

In certain embodiments, the substrate is an electrical conductor. In further embodiments, the electrical conductor is selected from the group consisting of a visible light transparent conductor, indium tin oxide (ITO), fluoride doped tin oxide (FTO), and polyethylene-terephthalate indium tin oxide (PET-ITO). In yet further embodiments, the electrical conductor is a visible light transparent conductor, optionally wherein the visible light transparent conductor is glass. In still further embodiments, the electrical conductor is indium tin oxide (ITO). In certain embodiments, the electrical conductor is fluoride doped tin oxide (FTO). In further embodiments, the electrical conductor is polyethylene-terephthalate indium tin oxide (PET-ITO).

In yet further embodiments, the solvent is a polar solvent. In still further embodiments, the polar solvent is a polar aprotic solvent. In certain embodiments, the polar aprotic solvent is dimethyl sulfoxide (DMSO).

In further embodiments, the first mixture has a concentration of a compound of the present disclosure of about 0.1 mg/mL to about 100 mg/mL. In yet further embodiments, the first mixture has a concentration of the compound of the present disclosure of about 1-10 mg/mL. In still further embodiments, the first mixture has a concentration of the compound of the present disclosure of about 5 mg/mL.

In certain embodiments, the treated substrate is heated to a temperature from about 50° C. to about 200° C. In further embodiments, the treated substrate is heated to a temperature from about 75-125° C. In yet further embodiments, the treated substrate is heated to a temperature of about 100° C. In still further embodiments, the treated substrate is heated for a time from about 1 minute to about 60 minutes. In certain embodiments, the treated substrate is heated for a time from about 5 minutes to about 30 minutes. In further embodiments, the treated substrate is heated for a time of about 15 minutes.

In still further aspects, the present disclosure provides photovoltaic devices comprising:
a substrate;
   an electron transport layer comprising a compound of the present disclosure;
   an active layer;
an interface layer;
a hole transport layer; and
a metal layer.

In certain embodiments, the active layer is a light absorbing active layer. In further embodiments, the active layer is a charge transporting active layer. In yet further embodiments, the active layer is an inorganic active layer. In still further embodiments, the inorganic active layer comprises a perovskite, a Dion-Jacobson phase, a Ruddlesden-Popper phase, or an Aurivillius phase. In certain embodiments, the inorganic active layer comprises a perovskite.

In further embodiments, the active layer is an organic active layer. In yet further embodiments, the organic active layer comprises a conjugated polymer. In still further embodiments, the substrate is an electrical conductor. In certain embodiments, the electrical conductor is selected from the group consisting of a visible light transparent conductor, indium tin oxide (ITO), fluoride doped tin oxide (FTO), and polyethylene-terephthalate indium tin oxide (PET-ITO). In further embodiments, the electrical conductor is a visible light transparent conductor, optionally wherein the visible light transparent conductor is glass. In yet further embodiments, the electrical conductor is indium tin oxide (ITO). In still further embodiments, the electrical conductor is fluoride doped tin oxide (FTO). In certain embodiments, the electrical conductor is polyethylene-terephthalate indium tin oxide (PET-ITO).

In further embodiments, the active layer comprises CsₓFA₁₋ₓPbI_{y}, wherein 0<x<0.30, 3<y<1.5, and FA is formamidinium. In yet further embodiments, the active layer comprises Cs_{0.09}FA_{0.91}PbI₃. In still further embodiments, the active layer is disposed along at least one surface of the substrate.

In certain embodiments, the interface layer comprises an organoammonium salt, an organoamine, a diamine, or a diammonium salt. In further embodiments, the interface layer comprises phenethylammonium iodide (PEAI). In yet further embodiments, the interface layer is disposed along at least one surface of the active layer.

In still further embodiments, the hole transport layer comprises poly(triarylamine) (PTAA), N4,N4,N4″,N4″-tetra([1,1′-biphenyl]-4-yl-[1,1′:4′,1″-terphenyl]-4,4″-diamine (TaTm), or 2,2′,7,7′-tetrakis[N,N-di(4-methoxyphenyl)amino]-9,9′-spirobifluorene (spiro-OMeTAD). In certain embodiments, the hole transport layer further comprises one or more dopants. In further embodiments, the one or more dopants are selected from the group consisting of lithium bis(trifluoromethane)sulfonate (Li-TFSI), 4-tert-butylpyridine, and tris(2-(1H-pyrazol-1-yl)-4-tert-butylpyridine)cobalt(III) tri[bis(trifluoromethane)sulfonimide](FK 209 Co(III)). In yet further embodiments, the hole transport layer is disposed along at least one surface of the interface layer.

In still further embodiments, the metal layer comprises a Group 11 or Group 13 element. In certain embodiments, the Group 11 or Group 13 element is selected from the group consisting of aluminum, copper, silver, and gold. In further embodiments, the Group 11 or Group 13 element is gold. In yet further embodiments, the metal layer is disposed along at least one surface of the hole transport layer.

In still further embodiments, the photovoltaic device further comprises an oxide layer. In certain embodiments, the oxide layer comprises titanium dioxide or tin dioxide. In further embodiments, the oxide layer is disposed between the substrate layer and the active layer.

In yet further embodiments, the photovoltaic device has an arithmetic mean power conversion efficiency (PCE) from about 1-20%. In still further embodiments, the photovoltaic device has an arithmetic mean PCE from about 4-11%. In certain embodiments, the photovoltaic device has an arithmetic mean PCE from about 6-9%. In further embodiments, the photovoltaic device has a maximum PCE from about 13-18%. In yet further embodiments, the photovoltaic device has a maximum PCE of about 15.5%.

In still further embodiments, the photovoltaic device has an arithmetic mean open circuit voltage (V_{OC}) from about 0.6-1.2 V. In certain embodiments, the photovoltaic device has an arithmetic mean open circuit voltage (V_{OC}) from about 0.8-1.0 V. In further embodiments, the photovoltaic device has a maximum open circuit voltage (V_{OC}) of about 0.99 V.

In yet further embodiments, the photovoltaic device has an arithmetic mean short-circuit current density (J_{SC}) from about 18-26 mA·cm⁻². In still further embodiments, the photovoltaic device has an arithmetic mean short-circuit current density (J_{SC}) from about 20-24 mA·cm⁻². In certain embodiments, the photovoltaic device has an arithmetic mean short-circuit current density (J_{SC}) from about 22-24 mA·cm⁻². In further embodiments, the photovoltaic device has an arithmetic mean short-circuit current density (J_{SC}) of about 24 mA·cm⁻². In yet further embodiments, the photovoltaic device has a maximum short-circuit current density (J_{SC}) of about 25 mA·cm².

In still further embodiments, the photovoltaic device has an arithmetic mean fill factor (FF) from about 40-80%. In certain embodiments, the photovoltaic device has an arithmetic mean fill factor (FF) from about 55-70%. In further embodiments, the photovoltaic device has an arithmetic mean fill factor (FF) of about 62%. In yet further embodiments, the photovoltaic device has a maximum fill factor (FF) of about 68%.

In still further embodiments, the photovoltaic device has an arithmetic mean stabilized maximum power point (MPP) from about 5-15%. In certain embodiments, the photovoltaic device has an arithmetic mean stabilized maximum power point (MPP) from about 7-10%. In further embodiments, the photovoltaic device has an arithmetic mean stabilized maximum power point (MPP) of about 8.9%. In yet further embodiments, the photovoltaic device has a maximum stabilized maximum power point (MPP) of about 13.5%.

In still further embodiments, the photovoltaic device is substantially thermally stable up to about 100° C. over a period of about 24 hours. In certain embodiments, the photovoltaic device is substantially thermally stable at about 90° C. over a period of about 12 hours. In further embodiments, the photovoltaic device is substantially photochemically stable to white light. In yet further embodiments, the photovoltaic device is substantially photochemically stable to ultraviolet light. In still further embodiments, the photovoltaic device is substantially inert to solvent.

In certain aspects, the present disclosure provides method of making a photovoltaic device, the method comprising:
contacting a substrate with a first solution comprising a compound of the present disclosure, thereby forming an electron transport layer disposed along at least one surface of the substrate;
contacting the electron transport layer with a second solution comprising an active layer precursor, thereby forming an active layer disposed along at least one surface of the electron transport layer;
contacting the active layer with a third solution comprising an interface compound, thereby forming an interface layer disposed along at least one surface of the electron transport layer;
contacting the interface layer with a fourth solution comprising a hole transport compound, thereby forming a hole transport layer disposed along at least one surface of the interface layer;
contacting the hole transport layer with a metal, thereby forming a metal layer disposed along at least one surface of the hole transport layer;
thereby forming the photovoltaic device.

In certain embodiments, the substrate is selected from the group consisting of a visible light transparent conductor, indium tin oxide (ITO), fluoride doped tin oxide (FTO), and polyethylene-terephthalate indium tin oxide (PET-ITO). In further embodiments, the substrate is a visible light transparent conductor, optionally wherein the visible light transparent conductor is glass. In yet further embodiments, the substrate is indium tin oxide (ITO). In still further embodiments, the substrate is fluoride doped tin oxide (FTO). In certain embodiments, the substrate is polyethylene-terephthalate indium tin oxide (PET-ITO).

In further embodiments, the method further comprises contacting the substrate with an oxide solution comprising an oxide, thereby forming an oxide layer disposed along at least one surface of the substrate; and further wherein the oxide layer is deposited before contacting the substrate with the first solution. In yet further embodiments, the oxide is titanium dioxide or tin dioxide. In still further embodiments, the oxide layer is deposited by spray pyrolysis.

In certain embodiments, the method further comprises a first annealing step following deposition of the oxide layer. In further embodiments, the first annealing step is performed at about 450° C. for about 30 minutes. In yet further embodiments, the electron transport layer is deposited by thermal evaporation. In still further embodiments, the thermal evaporation is performed at about 100° C. for about 15 minutes.

In certain embodiments, the active layer is a light absorbing active layer. In further embodiments, the active layer is a charge transporting active layer. In yet further embodiments, the active layer is an inorganic active layer. In still further embodiments, the inorganic active layer comprises a perovskite, a Dion-Jacobson phase, a Ruddlesden-Popper phase, or an Aurivillius phase. In certain embodiments, the inorganic active layer comprises a perovskite.

In further embodiments, the active layer is an organic active layer. In yet further embodiments, the organic active layer comprises a conjugated polymer. In still further embodiments, the active layer comprises CsₓFA₁₋ₓPbI_{y}, wherein 0<x<0.30, 3<y<1.5, and FA is formamidinium. In certain embodiments, the active layer comprises Cs_{0.09}FA_{0.91}PbI₃, wherein FA is formamidinium.

In further embodiments, the active layer is deposited by spin coating. In yet further embodiments, the spin coating is performed at about 1000 rpm for about 10 seconds, followed by about 6000 rpm for about 20 seconds.

In still further embodiments, the method further comprises a second annealing step following deposition of the active layer. In certain embodiments, the second annealing step is performed at about 150° C. for about 10 minutes.

In further embodiments, the interface layer comprises an organoammonium salt, an organoamine, a diamine, or a diammonium salt. In yet further embodiments, the interface layer comprises phenethylammonium iodide (PEAI). In still further embodiments, the interface layer is deposited by spin coating. In certain embodiments, the spin coating is performed at about 5000 rpm for about 20 seconds with an acceleration of about 5000 rpm/second.

In further embodiments, the method further comprises a third annealing step following deposition of the interface layer. In yet further embodiments, the third annealing step is performed at about 100° C. for about 10 minutes.

In still further embodiments, the hole transport layer comprises poly(triarylamine) (PTAA), N4,N4,N4″,N4″-tetra([1,1′-biphenyl]-4-yl-[1,1′:4′,1″-terphenyl]-4,4″-diamine (TaTm), or 2,2′,7,7′-tetrakis[N,N-di(4-methoxyphenyl)amino]-9,9′-spirobifluorene (spiro-OMeTAD). In certain embodiments, the hole transport layer further comprises one or more dopants selected from the group consisting of lithium bis(trifluoromethane)sulfonate (Li-TFSI), 4-tert-butylpyridine, and tris(2-(1H-pyrazol-1-yl)-4-tert-butylpyridine)cobalt(III) tri[bis(trifluoromethane)sulfonimide](FK 209 Co(III)). In further embodiments, the hole transport layer is deposited by spin coating. In yet further embodiments, the spin coating is performed dynamically at about 3000 rpm for about 30 seconds with an acceleration of about 3000 rpm/second.

In still further embodiments, the method further comprises a cleaning step following deposition of the hole transport layer. In certain embodiments, the cleaning step comprises contacting the substrate with dimethyl formamide and acetonitrile.

In further embodiments, the metal layer is deposited by thermal evaporation. In yet further embodiments, the metal is aluminum, copper, silver, or gold. In still further embodiments, the metal is gold. In certain embodiments, about 50 nm of gold is deposited.

In further aspects, the present disclosure provides organic light-emitting diodes (OLEDs) comprising a thin film of the present disclosure.

In yet further aspects, the present disclosure provides photodetectors comprising a thin film of the present disclosure.

In still further aspects, the present disclosure provides organic field-effect transistors (OFETs) comprising a compound of the present disclosure.

Perovskite solar cells (PSCs) have exhibited a rapid increase in power conversion efficiency (PCE) since their inception over a decade ago. The excitement around PSCs has been bolstered by their rapid advancement and the promise of low-cost and scalable fabrication. The electron transport layer (ETL) is an essential component of a high-performing PSC that facilitates the extraction and transport of electrons from the perovskite layer to the electrode while simultaneously blocking holes. Traditionally, inorganic ETLs such as TiO₂ and SnO₂ have been used but recent focus has shifted towards organic ETLs due to their low processing temperatures, chemical adaptability and compatibility with thermal evaporation.

However, there are few reports on the development of new organic ETLs that can be processed via both solution and evaporation. The most commonly used organic ETLs are fullerene-based materials, such as C₆₀ and [6,6]-phenyl-C₆₁-butyric acid methyl ester (PCBM). Despite advantageous charge transport properties, these materials suffer from poor frontier orbital tunability, high synthetic costs and poor ambient stability which has motivated the development of non-fullerene based organic-ETLs. Naphthalene diimide (NDI) and its derivatives provide an alternative route to achieving high efficiency PSCs that incorporate organic ETLs. NDIs have been extensively used in organic electronics owing to their electron affinity, high electron mobilities, tunable optical absorption profiles, and exceptional thermal stability. The strong electron withdrawing nature of imide functionalities results in NDIs possessing an electron deficient conjugated core, allowing it to form relatively stable radical anions. Cyclic voltammetry or a combination of ultraviolet photoelectron spectroscopy and UV-Vis spectroscopy have been used to show that NDI materials exhibit a deep lying LUMO position, often within close proximity to the perovskite conduction band. This allows NDI based materials to act as n-type organic semiconductors making them a prime candidate for use as organic-ETLs. The structural versatility of NDI molecules has spurred extensive research, leading to an array of derivative molecules functionalized at both the axial and terminal positions, as well as the integration of NDI subunits into polymers.

A key challenge limiting the widespread integration of NDI-based materials into conventional PSC architectures is the poor solubility of the parent NDI, which has terminal hydrogens on the imide nitrogen (NDI-H). One strategy to address the insolubility in common processing solvents leverages the structural tunability of NDIs by introducing solubilizing side groups. These structural modifications have been found to be effective in enhancing solubility, but some functionalization attempts, such as axial tert-butoxycarbonyl groups have been found to disrupt the optimal face-on stacking of the NDI cores that is desired for efficient charge transport. Despite this, functionalization using fluorinated hydrocarbons or chiral groups has enabled devices containing NDI-based ETLs to be competitive with the state-of-the-art perovskite technologies.

While undoubtedly impressive results have been obtained for PSCs utilizing NDI-based ETLs deposited via spin coating, incompatibility with large area or textured substrates has driven research into alternative solvent-free deposition techniques, such as thermal evaporation. Thermal evaporation involves sublimation of precursor materials under high vacuum and circumvents the need for processing solvents. This technique enables uniform coating of thin films over large areas and with precise control over film thickness and stoichiometry, while being well-suited for industrial scale-up. However, not all organic materials are compatible with the thermal evaporation process, as sufficient thermal stability is required at the elevated temperatures required for sublimation. It has been demonstrated that on occasion functional groups can render a material that is otherwise stable to evaporation unstable. A notable example of this is that while C₆₀ is readily sublimed, PCBM undergoes multiple decomposition routes when heated in ultra-high vacuum, resulting in thermally evaporated films containing PCBM, C₆₀, and other decomposition products. Limited research has been conducted on the thermal evaporation of NDIs for PSCs. Previously, unfunctionalized NDI-H was thermally evaporated as an ETL, achieving power conversion efficiencies of 10%, and outperforming the solution processed analog. Currently, there are few reports on how NDI functionalization influences the stability, structure, and performance of thermally evaporated NDI thin films. Gaining a deeper understanding of this relationship is essential for bridging the performance gap between spin coated and evaporated NDI films and could serve as a crucial stepping stone in achieving high performance, scalable PSCs.

The present disclosure provides a novel set of seven NDI derivatives with terminal functional groups designed to explore the impact of functional groups on the processability of NDI molecules via thermal evaporation. Of the seven synthesized molecules, two NDI molecules were down-selected: one molecule functionalized with the commonly-used phosphonic acid group ethyl phosphonic acid (NDI-dPA; also referred to as NDI-(EtPA)₂) and another with 4-bromophenyl (NDI-dBr; also referred to as NDI-(PhBr)₂) terminations. The former is expected to form strong interactions with the oxide substrate, whereas the latter is not. The molecule selection rationale is directed to understanding how terminal groups in these small molecules affect their deposition via thermal evaporation. The NDI-(PhBr)₂ was found to be stable during thermal evaporation, while NDI-(EtPA)₂ was shown to be unstable, which resulted in chemical changes during evaporation. The chemical composition and structure of both films was gauged via x-ray photoelectron spectroscopy (XPS), Fourier transform infrared spectroscopy (FTIR), and grazing incidence wide angle x-ray scattering (GIWAXS). NDI-(PhBr)₂ was shown to maintain its molecular structure during evaporation, while structural changes were observed in NDI-(EtPA)₂. Despite the observed thermal instability, structural analysis confirmed that NDI cores were still present in the evaporated NDI-(EtPA)₂ films. Optical absorption measurements demonstrated that both films possessed a suitable large bandgap for use as an ETL in an n-i-p device. The sensitivity of the NDI films to processing solvents used during perovskite deposition was also investigated. It was found that, for both molecules, a sufficient amount of material remained after exposure to polar solvents, which enabled them to act as effective ETLs. Finally, devices were fabricated to test the performance and long-term stability of NDI-(PhBr)₂ and NDI-(EtPA)₂. The devices exhibited PCEs of 15.6% and 14.1% for NDI-(PhBr)₂ and NDI-(EtPA)₂, respectively. Additionally, these devices were shown to be stable to environmental stressors such as heat, oxygen, and ultraviolet light and maintained a stable PCE over an operational period of 150 hours.

The present disclosure provides a family of NDI-derivative molecules, two of which were shown to act as effective electron transport layers in perovskite solar cells. In summary, it was found that ethyl phosphonic acid functional groups have a lower temperature threshold for thermal decomposition, which complicates their thermal evaporation. FTIR and XPS showed that the molecular structure of NDI-(PhBr)₂ is consistent between thermally evaporated, and solution processed films while NDI(EtPA)₂ undergoes chemical changes on evaporation. Despite these chemical changes, it was shown that NDI cores are present in both thermally evaporated films. Optoelectronic properties were probed, and it was found that the thermally evaporated NDI thin films possessed ideal large bandgaps. It was demonstrated that while both NDI-(EtPA)₂ and NDI-(PhBr)₂ thin films are sensitive to processing solvents used for perovskite deposition, the layers are not completely removed during the deposition process and still preserve their electron transport properties and charge selectivity. This enabled devices to be fabricated using thin films of both NDI-(EtPA)₂ and NDI-(PhBr)₂ with efficiencies of 14.1% and 15.6%, respectively. Both NDI-derivatives resulted in devices that outperform previous attempts at thermally evaporated NDI ETLs. Finally, we demonstrated the thermal and structural stability of our ETLs at operating conditions, and fabricated devices that demonstrate outstanding stability over 150 hours of continuous operation Therefore, this work not only establishes new efficiency records for thermally evaporated NDI thin films in ETL applications, but also identifies key synthetic considerations that are required for the design of scalable organic ETLs in the future.

### Definitions

Unless otherwise defined herein, scientific and technical terms used in this application shall have the meanings that are commonly understood by those of ordinary skill in the art. Generally, nomenclature used in connection with, and techniques of, chemistry, materials science, and electrical engineering described herein, are those well known and commonly used in the art.

The methods and techniques of the present disclosure are generally performed, unless otherwise indicated, according to conventional methods well known in the art and as described in various general and more specific references that are cited and discussed throughout this specification.

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), as well as macromolecules (such as conjugated polymers). Agents include, for example, agents whose structure is known, and those whose structure is not known.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “alkoxy” refers to an alkyl group having an oxygen attached thereto. Representative alkoxy groups include methoxy, ethoxy, propoxy, tert-butoxy and the like. The term “alkoxylene”, as used herein, refers to an alkoxy radical.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The term “heteroatom” as used herein means an atom of any element other than carbon or hydrogen. Preferred heteroatoms are nitrogen, oxygen, and sulfur.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “phosphate” as used herein is art recognized and refers to derivatives of the group PO₄³⁻. Phosphate may additionally refer to organophosphates of the form —R—O—P(═O)(O R′)(OR″), wherein R, R′, and R″ are organic groups.

The term “phosphonate” as used herein is art recognized and refers to a compound containing the group —R¹-P(═O)(OR²)(OR³), wherein R¹ is an organic radical, such as alkylene, and R² and R³ are each independently hydrogen or an organic group, such as an alkyl group.

### EXAMPLES

The invention now being generally described, it will be more readily understood by reference to the following examples which are included merely for purposes of illustration of certain aspects and embodiments of the present invention, and are not intended to limit the invention.

### Example 1: Exemplary Molecular Synthesis and Thermal Stability of Compounds and Thin Films of this Disclosure

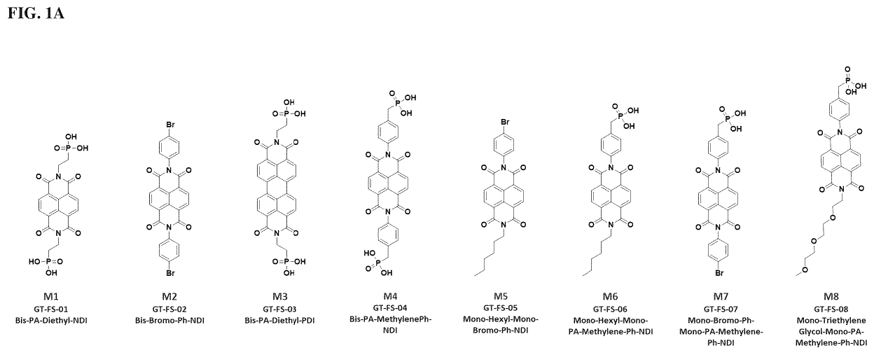

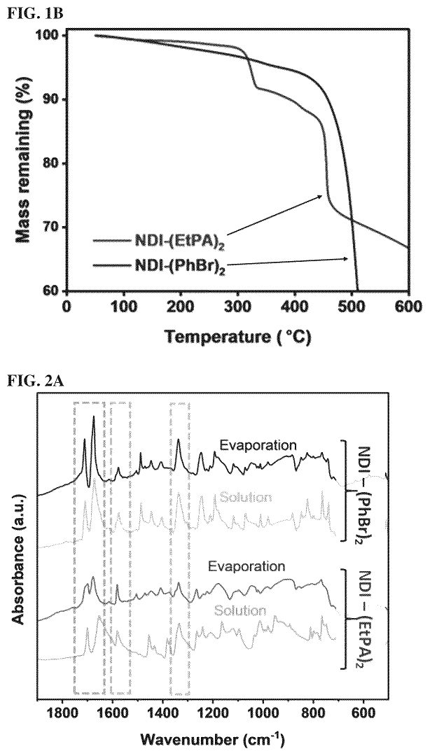

Molecular Synthesis and Thermal Stability To enable ETL deposition via thermal evaporation, it was essential to identify a set of molecules that not only met the key requirements for an effective ETL in a PSC but also possessed the thermal stability necessary for successful evaporation. A group of NDI derivatives were synthesized and functionalized only at the terminal imide positions, as shown in FIG. 1A. The success of each synthetic step and purity of the final products were confirmed via ¹H, ¹³C, and ³¹P Nuclear Magnetic Resonance (NMR) spectroscopy. Functionalization with Ethyl-phosphonic acid was chosen for its ability to form robust covalent bonds with oxide substrates, promoting strong adhesion and minimizing delamination. Additionally, previous research has shown that phosphonic acids can induce the growth of cubic formamidinium (FA) based perovskites phases, leading to improvements in device performance. Aliphatic hexyl chains were selected to improve solubility by virtue of their conformational flexibility, which allows for easier synthesis, purification, and isolation. Aryl-bromide groups were selected to enhance the solubility relative to NDI-H, however, they offer greater rigidity than alkyl chains, which could improve thermal stability. The hydrophobic character of the bromophenyl and hexyl chains, relative to ethyl phosphonic acid, could potentially also enhance long-term stability under humid conditions.

This work introduces seven NDI derivatives, functionalized at the terminal positions, for use as thermally ETLs in PSCs. Two especially well-performing NDI derivatives were discovered. NDI-(EtPA)₂ (left) was functionalized with an ethyl phosphonic acid group on both terminal positions while NDI-(PhBr)₂ (right) was functionalized with phenyl bromide groups in the terminal positions. The compiled structures of all NDI derivative molecules synthesized in this work can be found in Table 2. NDI 1 is referred to as NDI-1, NDI-(EtPA)₂, or NDI-dPA and NDI 2 is referred to as NDI-2, NDI-(PhBr)₂, or NDI-dBr throughout the application. The synthetic routes used to synthesize all molecules can be found within the synthetic protocol section of the examples section. Characterization via mass 1H NMR was employed to confirm the success of these syntheses and ensure the purity of the resulting products. This data is also shown in the synthetic protocol section of the examples section.

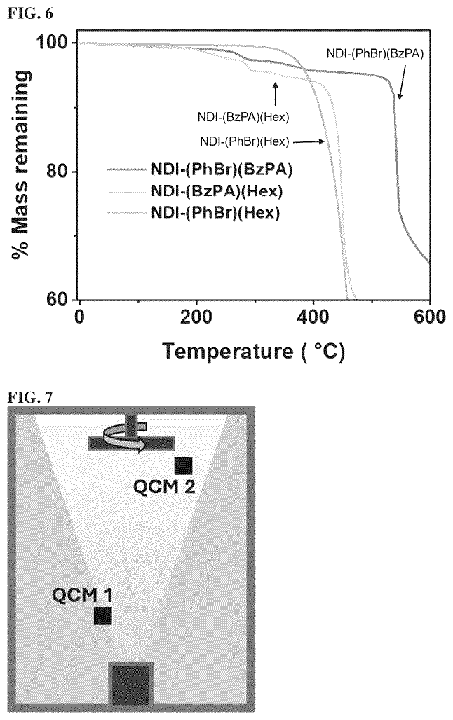

Given the focus on the thermal evaporation of the NDI molecules, understanding their thermal stability is crucial. For a molecule to undergo complete evaporation without decomposition, sufficient thermal energy must be provided to overcome the intermolecular forces present while avoiding bond cleavage and thermal degradation. Thermogravimetric analysis (TGA) provides valuable insights into a material's suitability for evaporation by highlighting the thermal instabilities of a material. FIG. 1B shows the TGA scans for both NDI-(EtPA)₂ and NDI-(PhBr)₂ under nitrogen atmosphere, while thermograms for the other NDI derivatives are provided in FIG. 6. For NDI-(PhBr)₂ (FIG. 1B) and NDI-(PhBr)(Hex), which lack phosphonic acid functionalization, a single distinct mass loss event occurs with an onset temperature of approximately 440° C. In contrast, the thermogram of NDI-(EtPA)₂ (FIG. 1B) exhibits a more complex profile, showing an initial mass loss of approximately 10% at 315° C. followed by a second mass loss event at around 440° C. Given that neither of these mass loss events result in complete volatilization, they likely correspond to some decomposition process, either via the expulsion of volatile decomposition products or the formation of side products within the material. A purely evaporative process would be characterized by a large single-step mass loss. Similar multiple-step mass loss behavior is observed for NDI(PhPA)(Hex) and NDI(PhPA)(PhBr) (FIG. 6), indicating that this instability is an inherent characteristic of NDI molecules functionalized with phosphonic acid groups. Furthermore, the similarity in the thermograms of NDI-(EtPA) and NDI-(PhPA)(PhBr) suggests that replacing the ethyl linker with a benzyl linker does not significantly improve the thermal stability of the molecule. The observed thermal instability of PA-functionalized NDI molecules underscores the need for careful consideration of functional group selection when designing NDI derivatives for thermal evaporation processes.

### Example 2: Exemplary Structural Characterization of Evaporated Thin Films of this Disclosure

To further probe the thermal evaporation and subsequent performance of NDI-based ETLs, NDI-(PhBr)₂ and NDI-(EtPA)₂ were selected for further investigation as their distinct, yet different, thermal behaviors can provide valuable insight into the impact of different terminal substituents on the evaporation characteristics. The single, high-temperature mass loss event observed for NDI-(PhBr)₂ indicating clean evaporation with minimal decomposition makes it a strong candidate for thermal evaporation processing. In contrast, NDI-(EtPA)₂, while exhibiting multiple mass loss events, serve as a representative of the PA-functionalized NDIs, allowing for direct comparisons of how these functional groups influence stability. By focusing on these two symmetrically substituted molecules, the complexities introduced by asymmetry are minimized, facilitating a cleaner understanding of structure-property relationships critical for optimizing thermally evaporated ETLs.

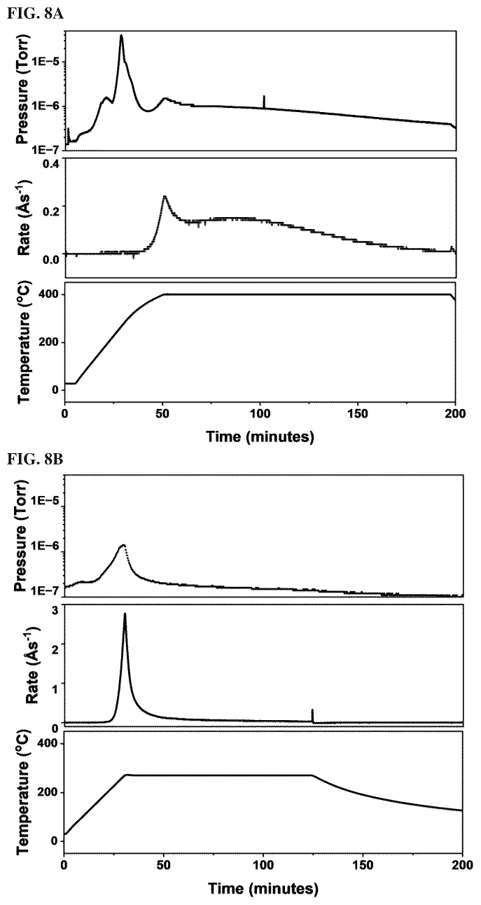

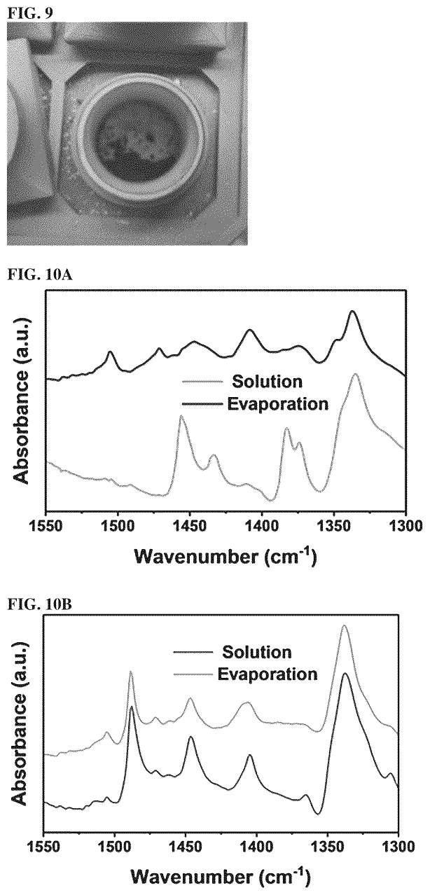

Thin films of the NDI transport layers were deposited via thermal evaporation on fluorine-doped tin oxide (FTO) coated glass, glass slides, and flexible poly(ethylene terephthalate) substrates coated with indium-doped tin oxide (PET:ITO). A schematic of the evaporation chamber used here can be seen in FIG. 7. Each evaporation was monitored via Quartz Crystal Microbalance (QCM) and experimental parameters such as pressure, deposition rate and temperature are shown in FIGS. 8A-8B. The evaporation of both molecules continued until QCM 1 stopped registering the deposition of fresh material. Both NDI-(PhBr)₂ and NDI-(EtPA)₂ exhibited distinct deposition events, as shown in FIGS. 8A-8B, at approximately 220° C. and 380° C., respectively. The elevated deposition onset temperature of NDI-(EtPA)₂ could be explained by the stronger intermolecular forces that arise from the phosphonic acid-induced hydrogen bonding. The deposition temperature of NDI-(PhBr)₂ was recorded to be below that of the first mass-loss event, which was shown to be approximately 440° C. This is a strong indication that NDI-(PhBr)₂ possesses the thermal stability to be thermally evaporated. Conversely, the thermogram of NDI-(EtPA)₂ in FIG. 1B exhibits a mass loss event at 315° C. This is below the temperature at which deposition was recorded, as shown in FIG. 8, which suggests that NDI-(EtPA)₂ does not possess the thermal stability to evaporate without undergoing some form of chemical change. After thermal deposition, no material remained in the crucible after the evaporation of NDI-(PhBr)₂, while residual material was found in the crucible containing NDI-(EtPA)₂, as shown in FIG. 9. In fact, for all molecules containing phosphonic acid functionalities, some degree of material remained in the crucible post deposition. This helps corroborate the hypothesis that phosphonic acid functional groups have a propensity to undergo some degree of chemical change during evaporation, when functionalized to an NDI core. While reports on thermally evaporated small molecule organic ETLs are sparse, thermally evaporated hole transport layers (HTLs) are better studied. One example is the [2-(9H-carbazole-9-yl)ethyl]phosphonic acid (2PACz) family of molecules, which consists of phosphonic acid-functionalized carbazoles. Thermal instability has been identified in this family of molecules, whereby degradation and residual powders were identified at temperatures above 200° C. The present findings, alongside these reports, lead to the conclusion that ethyl phosphonic acid functionalization introduces inherent thermal instability to certain small molecules. Therefore, emphasis should be placed on the future choice of the phosphonic acid-functionalized core molecule and the temperatures at which they are thermally evaporated to ensure that film deposition can occur before chemical changes happen to the parent molecule.

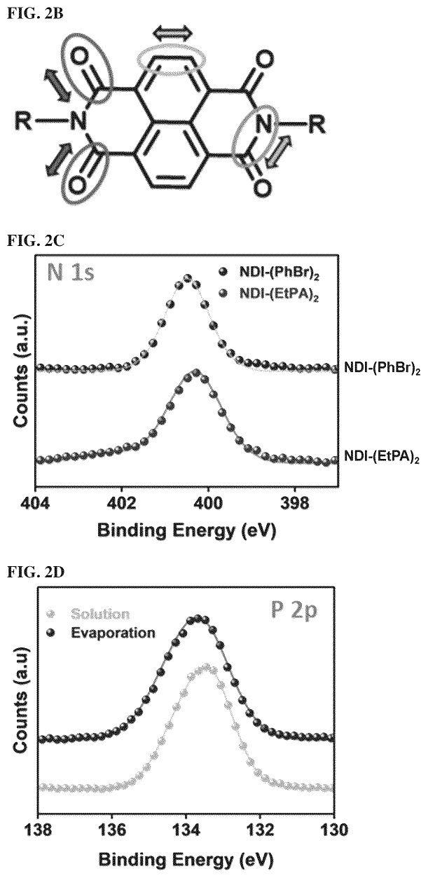

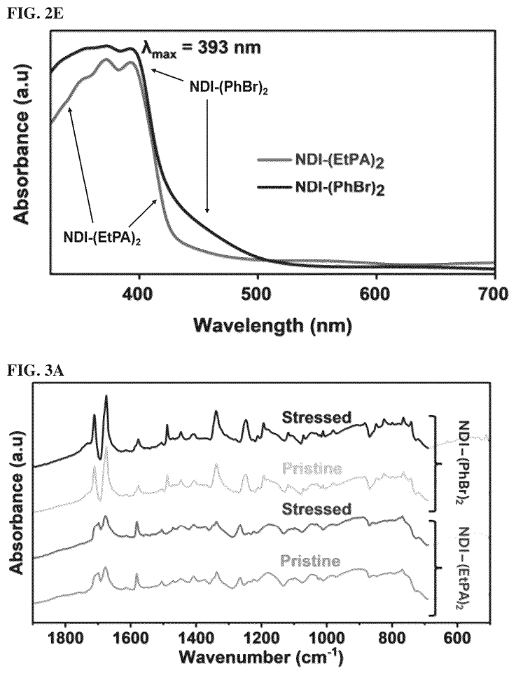

To identify changes in the chemical structure of these NDI molecules, we can compare thermally evaporated films to drop-cast films, given that no molecular changes are to be expected during room-temperature solution processing of the NDI molecules. Attenuated total reflectance Fourier transform infrared (FTIR) spectroscopy can help identify any chemical changes within a material by identifying changes in molecular vibrations, which relate to changing chemical environments and the cleavage/formation of chemical bonds. FIG. 2A shows the FTIR spectra for thermally evaporated and solution processed films of NDI-(EtPA)₂ and NDI-(PhBr)₂ on flexible PET:ITO substrates. The spectra for both evaporated and solution processed NDI-(PhBr)₂ share identical peak distributions, with some variation in peak intensity. This suggests that NDI-(PhBr>₂ is evaporated without thermal decomposition taking place. When considering the FTIR spectra of the evaporated and solution processed NDI-(EtPA)₂ thin films, distinct differences are evident. FIG. 10A provides an overlay of the FTIR spectra of both the solution processed and thermally evaporated thin films of NDI-(EtPA)₂ and NDI-(PhBr)₂ between the range of 1550 cm⁻¹ and 1300 cm⁻¹. Throughout this range several differences in peak intensities can be seen between the NDI-(EtPA)₂ spectra, signifying structural changes occurring during thermal evaporation. FIG. 2A compares FTIR of evaporated and solution deposited films which allow for more precision in understanding the chemical changes occurring during evaporation. First, a doublet is observed at around 1700 cm⁻¹ which can be attributed to the symmetric and asymmetric C═O stretches of the imide moiety. While the imide doublets exhibit similar features in the NDI-(PhBr)₂ spectra, a distinct change is seen in the NDI-(EtPA)₂ doublet. The thermally evaporated film shows a narrowing of the doublet split, alongside a distinct narrowing of the peak observed at lower wavenumbers which is associated with the asymmetric carbonyl stretch. This evolution, coupled with the more dramatic changes in FIG. 10A, suggest chemical changes have occurred, which disrupts the symmetry of NDI-(EtPA)₂. Additionally, a peak at approximately 1580 cm⁻¹ is observed in all spectra and highlighted via the green box in FIG. 2A. This can be attributed to the stretching vibration of the naphthalene ring skeleton. A peak is also observed in all spectra at 1335 cm⁻¹, as highlighted in the purple box in FIG. 2A. This is attributed to the C-N imide stretch. These characteristic vibrations are schematically presented in FIG. 2B and suggest that the core NDI-structure is mostly uncompromised during deposition via thermal evaporation for either molecule, along with suggesting that in the case of NDI-(EtPA)₂ the changes may be occurring on the EtPA moiety. Meanwhile the FTIR analysis supports the argument that NDI-(PhBr)₂ remains fully intact throughout the evaporation process.

X-Ray Photoelectron Spectroscopy (XPS) measurements were conducted on NDI films deposited on FTO to provide insights into the chemical bonding and environment present within the NDI films. FIG. 2C shows the N-1s XPS spectrum for thermally evaporated thin films of both NDI-(PhBr)₂ and NDI-(EtPA)₂. The emitted photoelectrons were detected at similar peak positions of 400.5 eV and 400.3 eV for NDI-(PhBr)₂ and NDI-(EtPA)₂, respectively. The proximity of these photoelectron peaks suggests that the majority of the nitrogen exists in the same imide environment for both samples, providing further evidence that the NDI core remains intact during the evaporation of either molecule. The small difference in binding energy can be ascribed to differing electronic environments around the nitrogen in NDI-(PhBr)₂ and NDI-(EtPA)₂ due to the presence of an sp² C-N bond and an sp³ C-N bond, respectively. Interestingly, a tail at higher binding energies can be observed for NDI-(EtPA)₂ that is not observed in the solution processed film shown in FIG. 11. While it is difficult to ascribe this tail to any particular species, its presence suggests that during the evaporation of NDI-(EtPA)₂ a minor product is formed in which the nitrogen exists in a more electron deficient environment. FIG. 2D shows the P 2p XPS spectra for NDI-(EtPA)₂ deposited via solution and thermal evaporation. Phosphorus is present on the surface of both samples, and the peak binding energies were found to be 133.5 eV and 133.6 eV, respectively. The presence of the P 2p XPS signals, along with previously discussed evidence of the NDI core being maintained, makes conclusions about the nature of the thermal instability difficult. While the apparent robustness of the NDI core could lead one to conclude that decomposition is due to the alkyl phosphonate functionality, cleavage of this moiety in its entirety is unlikely, given the relatively small mass loss observed in the early decomposition stage of the NDI-(EtPA)₂ thermogram that is shown in FIG. 1B.

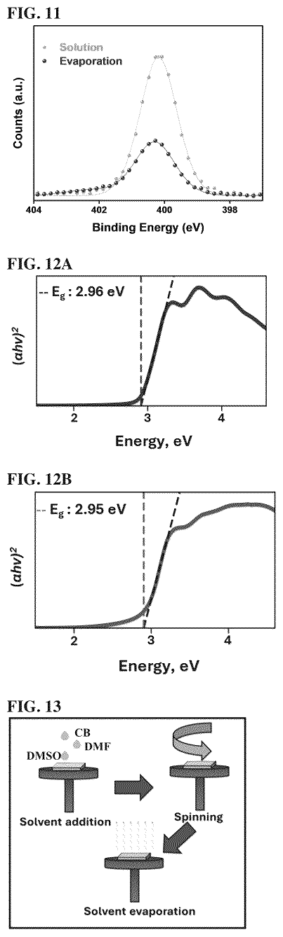

Further insights into the nature of the films deposited by thermal evaporation, and their suitability as charge transport layers, can be found through characterization of their optoelectronic properties. FIG. 2E shows the UV-vis absorption spectra for thin films of thermally evaporated NDI-(EtPA)₂ and NDI-(PhBr)₂. As expected, both molecules possess the same absorption peak with a maximum value of 393 nm. This matches the absorption values found in the literature for NDI-H and its derivatives attributed to the π-π* transition of the NDI core. This corroborates previous assertions from FTIR and XPS that the NDI core is being evaporated. NDI-(PhBr)₂ exhibits a lower-energy absorption onset than NDI-(EtPA)₂, with a gradual absorption increase beginning at approximately 500 nm, whereas NDI-(EtPA)₂ shows a sharp onset beginning at approximately 430 nm. It is possible that this gradual absorption onset, alongside the reduced vibronic features, is due to a more disordered film in the instance of NDI-(PhBr)₂ relative to NDI-(EtPA)₂. The optical absorption spectra also allow for determination of the optical band gap through the generation of Tauc plots (FIG. 12A). The bandgaps of NDI-(EtPA)₂ and NDI-(PhBr)₂ were found to be 2.96 eV and 2.95 eV, respectively. These band gaps are sufficiently wide to allow the transmission of visible and NIR light, which is vital for efficient ETLs to maximize the number of photons reaching the active layer and optimize the PCE in solar cells.

### Example 3: Exemplary Optoelectronic Characterization of Evaporated Thin Films of this Disclosure

Further insights into the nature of the films deposited, and their suitability as charge transport layers, can be found through characterization of their optoelectronic properties. FIG. 2E shows the UV-Vis absorption spectra for both NDI-dPA and NDI-dBr respectively between the range of 300-700 nm. Both molecules appear to possess the same absorption peak onset, with a maximum value of 393 nm. This closely matches the absorption values found in the literature for NDI-H and its derivatives. This corroborates previous assertions from FTIR and XPS that the NDI core is being evaporated, given that the HOMO-LUMO transition is prescribed to the π-π* transition of the NDI core. This onset of absorption wavelength of approximately 400 nm is shared by all the NDI molecules introduced in this paper, as shown in FIGS. 2E and 33.

UV-Vis spectra also allow for determination of the optical band gap through the generation of Tauc plots which are displayed in FIGS. 12A and 12B for NDI-dPA and NDI-dBr respectively. The band gaps for NDI-dPA and NDI-dBr were found to be 2.96 eV and 2.95 eV, respectively. In n-i-p devices, it is crucial for the electron transport layer (ETL) to possess a large band gap to minimize its absorption of incident light. Although the band gaps of the NDI derivatives are smaller than those of TiO₂ (2=3.2 eV) and SnO₂ (=3.6 eV), the NDI-derivative band gaps of =3 eV suggest that the NDI layers would still be transparent to most incident light. This is an indication that NDI derivatives are good candidates for use as effective ETLs.

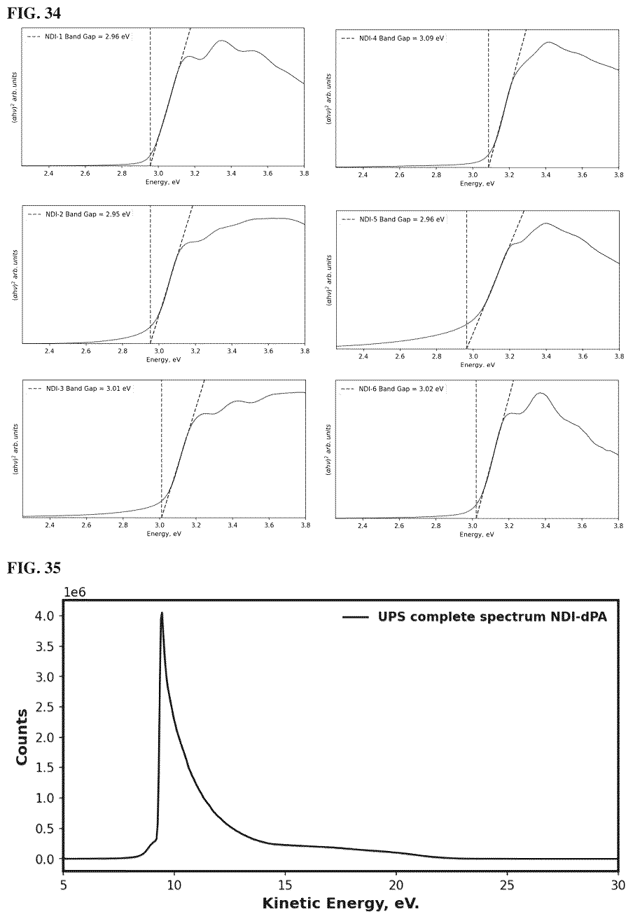

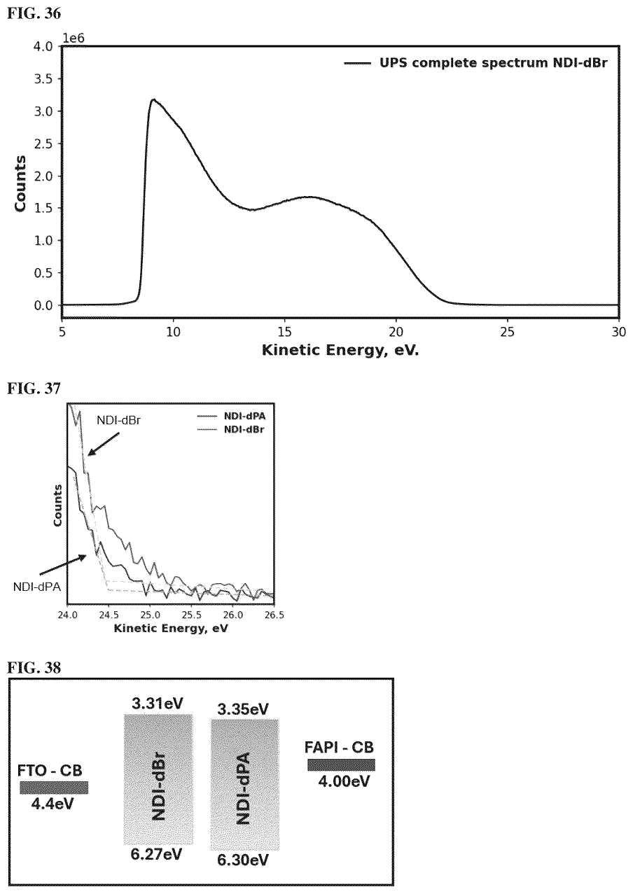

Ideally, the LUMO of the electron transporting layer should be slightly lower in energy (relative to vacuum) than that of the perovskite conduction band. The LUMO energy positions for NDI-dPA and NDI-dBr were calculated via UPS and Tauc plots to be 3.35 eV and 3.27 eV, respectively (FIGS. 34-38). This finding indicates that there could be marginal energetic misalignment at the perovksite/ETL interface, given that the perovskite composition used has a CBM of approximately 4 eV. While such misalignments are suboptimal, previous research has found, in some cases, that V_{OC} can be independent of band alignment. Additionally, in future work axial functionalization of the NDI molecule would allow for incorporation of EWGs directly into the conjugated system, enabling further stabilization of the radical anion and effective reduction of the LUMO energy.

### Example 4: Thin Film Stability in Operating Conditions

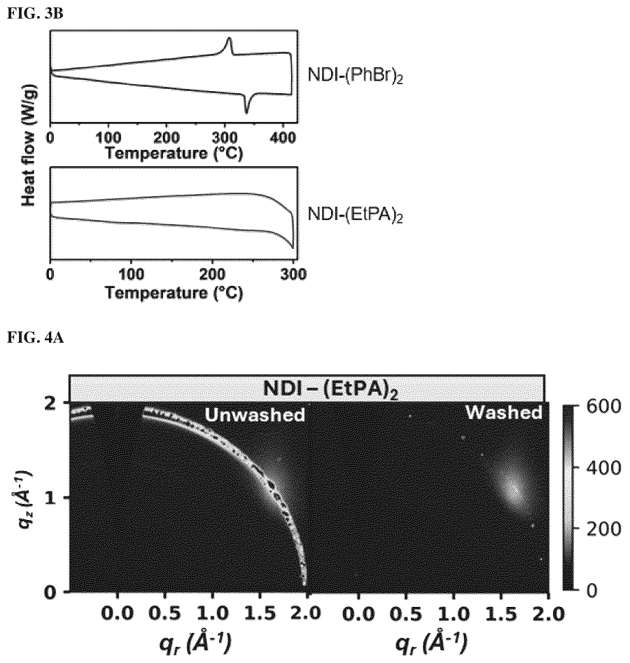

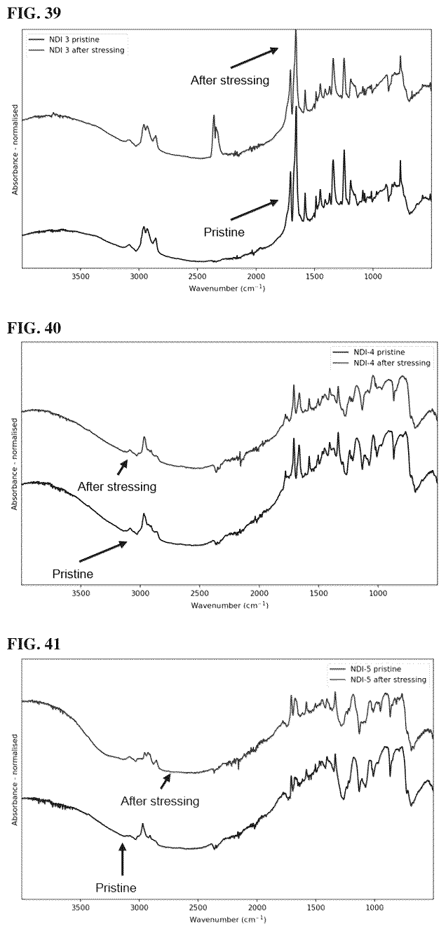

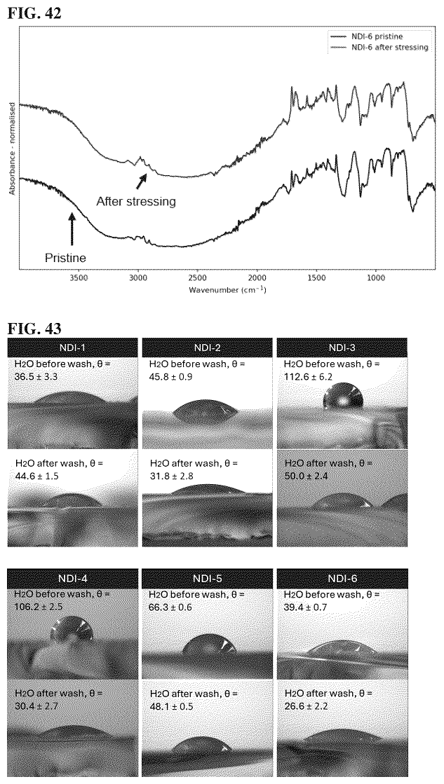

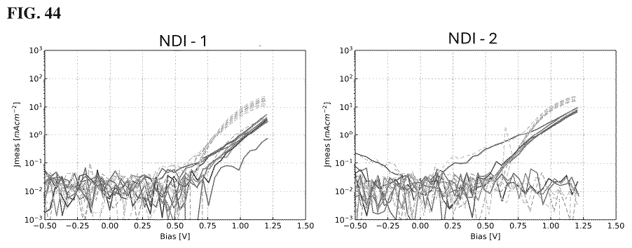

Thermochemical stability is a vital consideration for each layer of the device stack in a perovskite solar cell. Current testing standards require devices to exhibit stability at temperatures of 85° C., and hence it is important that all layers within a PSC is thermally robust at these elevated temperatures. Additionally, films should be able to withstand the synergetic effects of oxygen and UV light to minimize photooxidation. To test the thermal stability of such films in standard operating conditions, FTIR spectra were collected before and after stressing the NDI film at 90° C. for 12 hours in air (with around 60% relative humidity) under 1 sun equivalent white light. As can be seen from FIG. 3A there are no significant changes in the vibrational modes of the films before and after stressing. This suggests that these ETL films are structurally stable in ambient operating conditions. The pristine/stressed FTIR comparisons for the remaining NDI molecules can be found in FIGS. 39-42.

Despite the evident stability of these films under common operating conditions, consideration must also be given to the morphological stability of a material. Previously it has been reported that organic materials can undergo phase changes at elevated temperatures that can result in poor operational stability over long periods of time. Differential Scanning Calorimetry (DSC) can identify such thermal transitions through analysis of the heat flow as a sample is heated and cooled. Previous reports have shown that films which exhibit thermal transitions in their DSC scans at low temperatures are more prone to instability over longer periods of time. FIG. 3B displays the DSC thermograms for both NDI-(EtPA)₂ and NDI-(PhBr)₂ molecules. Heating both NDI-(EtPA)₂ and NDI-(PhBr)₂ induces recrystallization or melting events at temperatures of 340° C. and 300° C. respectively. Importantly, no obvious thermal transitions are observed at the temperatures associated with device fabrication or operation as the most extreme temperature the films would be subjected to is 150° C. during the perovskite deposition step. The combined findings of the FTIR spectra post stressing and DSC analysis support the conclusion that the NDI films are both chemically and structurally stable under operating conditions and should exhibit consistent performance over extended periods of time.

### Example 5: Sensitivity of Organic Transport Layers to Process Solvents

The sequential nature of perovskite device fabrication necessitates that NDI ETLs in n-i-p devices remain resistant to the processing solvents used in depositing subsequent layers, assuming those layers are deposited via spin-coating, the current standard in the field. Given that molecular thin films are often dominated by non-covalent interactions, processing solvents such as dimethylformamide (DMF) and dimethyl sulfoxide (DMSO) can disrupt intermolecular interactions, and hence solvent-film interactions should not be overlooked. Furthermore, the degree to which solvents interact with organic materials can often be impacted by the functional groups present. This behavior has been observed in NDIs whereby, for instance, functionalization with alkyl chains results in stronger solvent-NDI molecule interactions and potential dissolution. Therefore, it is possible that the differing nature of the ethyl phosphonic acid and aryl bromide functional groups would result in varying degrees of interaction with the processing solvents used.

Despite solvent free deposition techniques being the preferred method of thin film deposition for industry, currently the majority of perovskite research utilizes perovskite thin films deposited via solution-based techniques such as spin coating. Substrate-solvent interactions between process solvents and inorganic ETLs have received limited attention in the literature, largely due to the chemical and structural stability of these ETL layers when exposed to organic solvents. This stability does not necessarily extend to molecular thin films, given that certain organic ETLs have been shown to be particularly sensitive to processing solvents. Despite NDI being generally regarded as resistant to process solvents, functionalization can impact solubility and hence solvent-ETL interactions should not be discounted when introducing NDI derivatives.

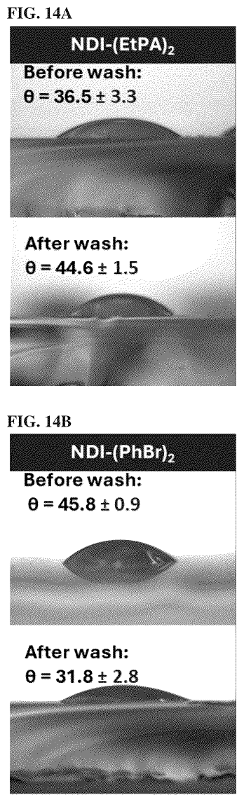

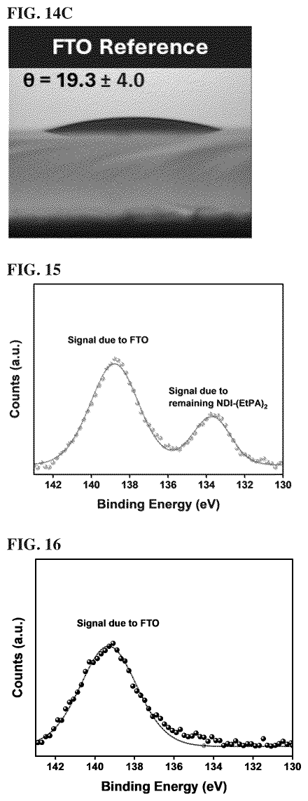

To test the sensitivity of our NDI ETLs to subsequent steps of device fabrication, a solvent wash was conducted. In this process, which is shown schematically in FIG. 13, the conditions present during perovskite deposition were simulated without any perovskite precursors to allow for the solvent-ETL interactions to be gauged without the formation of a perovskite phase. Initially, 90 μL of a 2:1 DMF:DMSO mixture was spin-coated onto the NDI films to replicate the perovskite deposition step. This was followed by spin-coating 250 μL of chlorobenzene, which acts as an antisolvent during perovskite deposition, before annealing at 150° C. for 10 minutes. Preliminary indication into the solvent sensitivity of our films was obtained through contact angle measurements, shown in FIG. 14A. Both film surfaces exhibit changes in the water contact angle on solvent washing, indicating some changes in surface polarity.

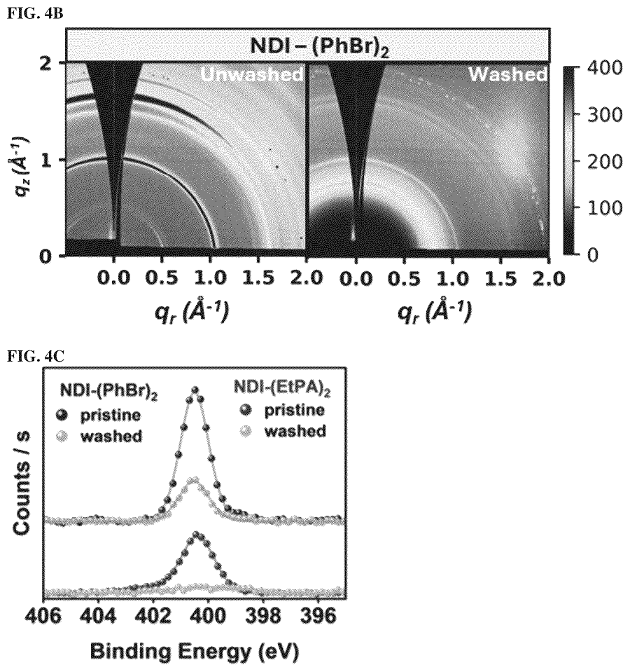

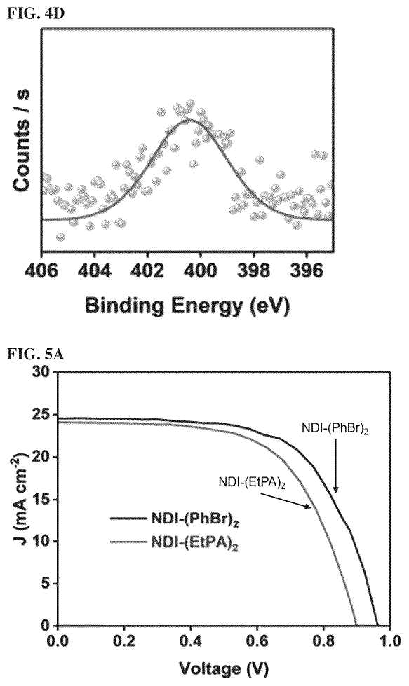

Synchrotron-based grazing incidence wide-angle X-ray scattering (GIWAXS) was utilized to further assess the impacts of solvent washing on the structure of the NDI films. FIG. 4A shows the two-dimensional scattering patterns of NDI-(EtPA)₂ films at an incident angle α=0.5° before and after washing. The unwashed NDI-(EtPA)₂ sample exhibits a Debye-Scherrer ring at q_{z}=2.0 Å⁻¹, which is indicative of crystallinity, increasing intensity around qr=0 Å⁻¹ suggests the material is also exhibiting some degree of preferential face-on orientation. We attribute this feature to ordering along the (112⁻) plane, which has been previously attributed to π-π stacking of the NDI cores. Upon washing, the Debye-Scherrer ring disappears almost entirely for the NDI-(EtPA)₂ film. This suggests that the washing process removes all crystalline features of the film. As NDI-(EtPA)₂ is polar and protic, it is expected to undergo favorable interactions with DMSO and DMF. The observed sensitivity to processing solvents is therefore not unexpected. The two-dimensional scattering patterns of NDI-(PhBr)₂ before and after washing are shown in FIG. 4B. The unwashed NDI-(PhBr)₂ sample exhibits a more complicated diffraction pattern, a characteristic that has been observed before in NDI based materials. A crystalline feature can be observed at qz=1.7 Å⁻¹, which is attributed to stacking in the (010) plane, a feature that the literature reports is also due to π-π stacking. The more intense diffraction around qr=0 Å⁻¹ implies preferential face-on orientation of the molecules. The presence of multiple features and an increase in amorphous background signal at higher q values point towards a film exhibiting crystallographic variation. This could help explain the tail and reduced vibronic features observed in the optical absorption of NDI-(PhBr)₂ which we previously attributed to local disorder. Upon washing, the features at qz=1.7 Å⁻¹ and qz=1.0 Å⁻¹ disappear and a new broad Debye-Scherrer ring appears at qz=0.6 Å⁻¹. This is evidence that while NDI-(PhBr)₂ is recrystallizing into a different structure on washing, a greater portion of the film remains than in the instance of NDI-(EtPA)₂.

To help strengthen any conclusions made about the nature of the films remaining after the solvent wash, XPS was conducted on films of NDI-(PhBr)₂ and NDI-(EtPA)₂ before and after washing. The N 1s spectra for both films are shown in FIG. 4C. While prominent N 1s signals are observed before washing for both films, significant reductions in signal intensity are observed after washing. For NDI-(PhBr)₂, although a clear reduction is observed, there remains a significant N is signal after washing. This corroborates our GIWAXS findings and allows us to conclude definitively that while NDI-(PhBr)₂ does experience interactions with processing solvents, a thin film remains on washing, albeit with a changed crystal structure. The N 1s signal for NDI-(EtPA)₂ is almost completely absent after solvent washing which corroborates the absence of crystalline features in the GIWAXS patterns. These findings suggest that the bulk portion of the NDI-(EtPA)₂ film is being removed. Despite the large reduction in intensity, a small N 1s signal can still be observed in the XPS spectrum, as shown in FIG. 4D. To ensure that this signal is not due to surface contamination, a P 2p spectrum is also shown in FIG. 15. A clear P 2p peak is evident at 134.1 eV, which aligns with the peak observed in the unwashed sample. Another peak at 139.2 eV can also be observed in the spectrum. This peak is assigned to the FTO given that this peak is observed in the XPS spectrum of bare FTO, shown in FIG. 16.

### Example 6: Exemplary Device Performance

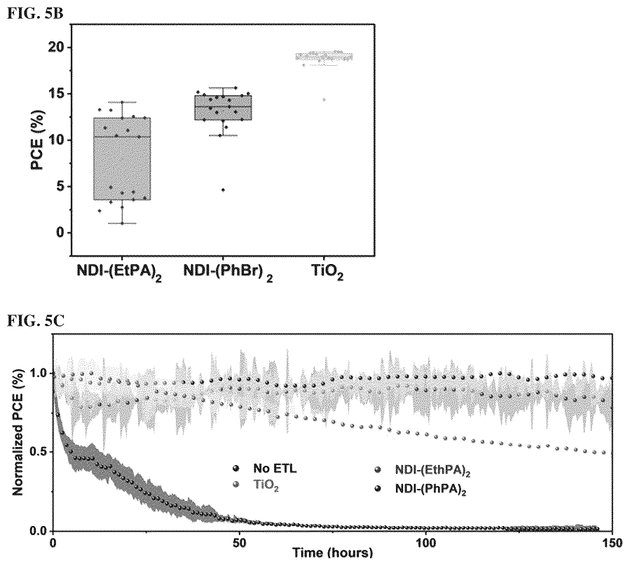

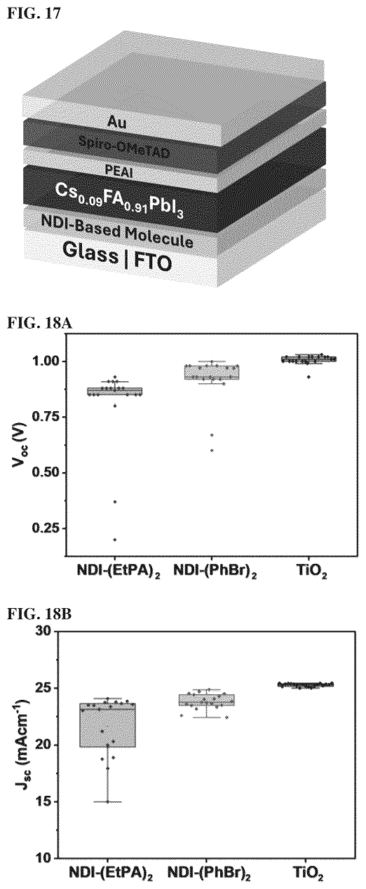

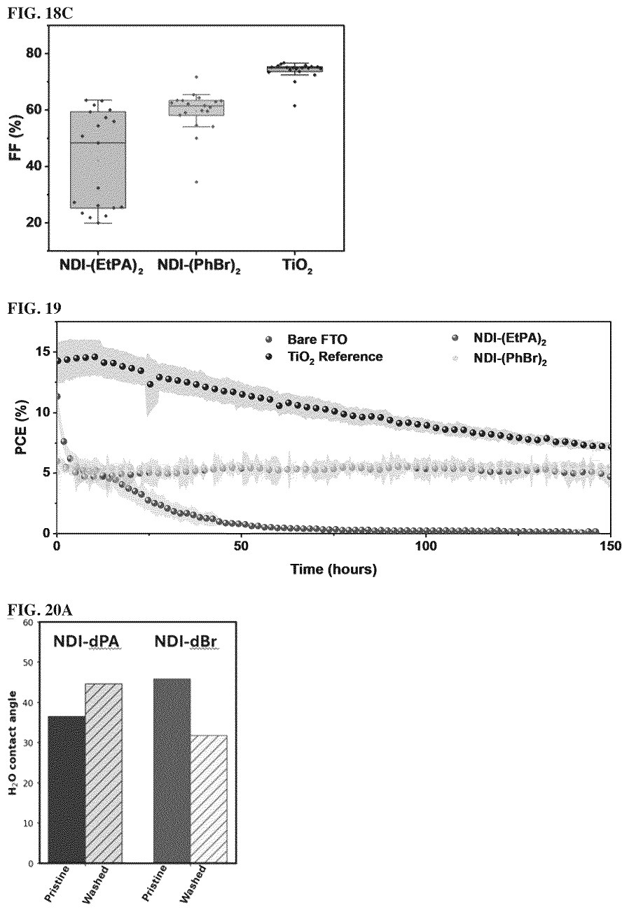

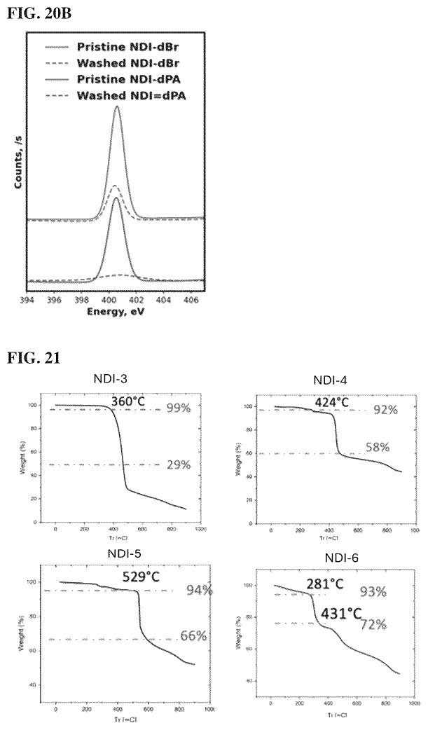

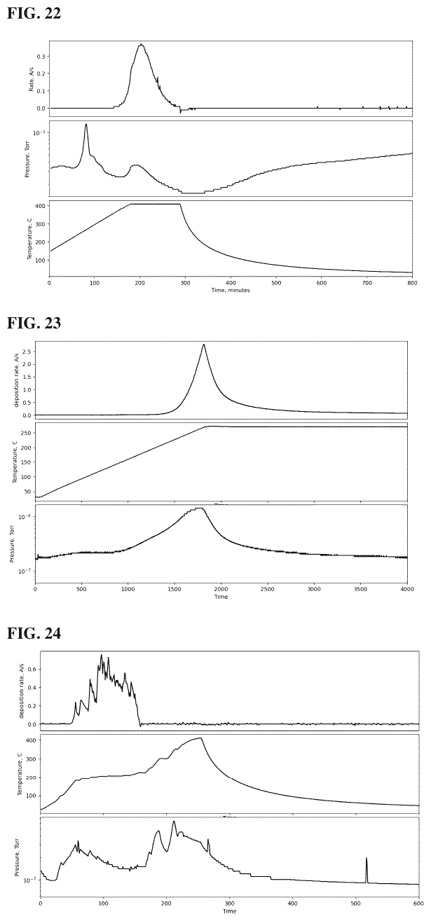

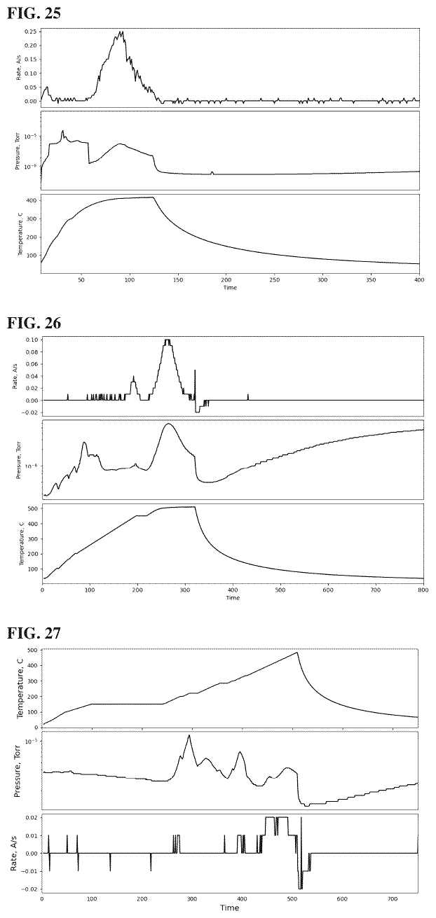

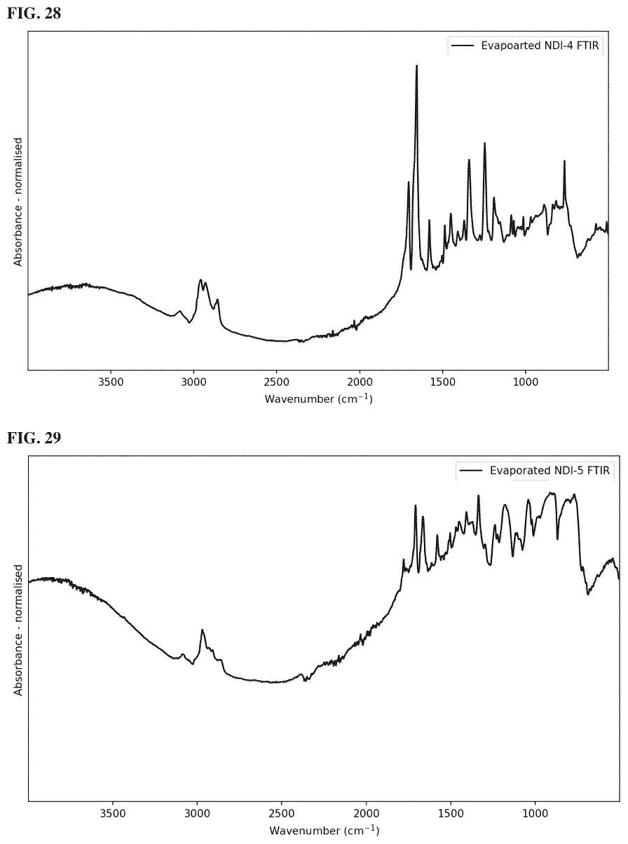

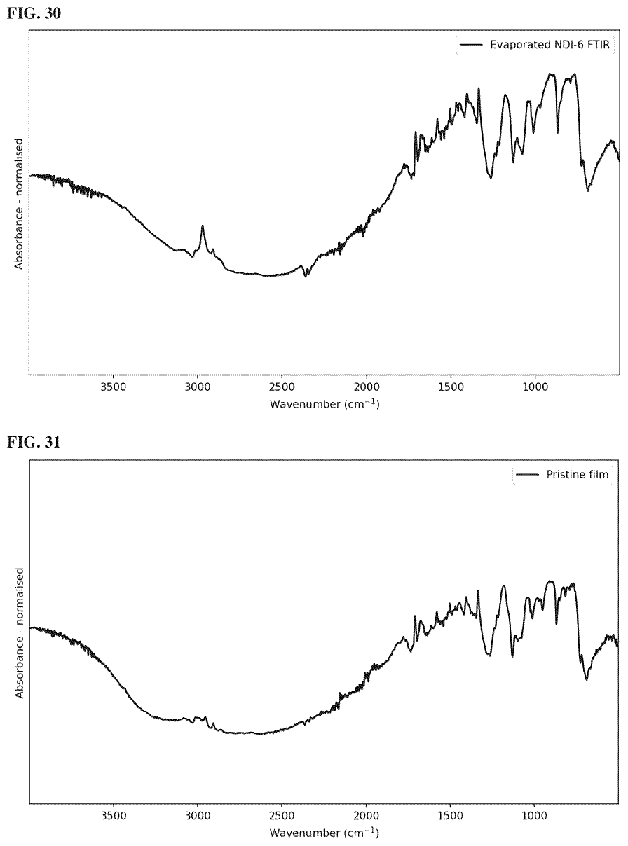

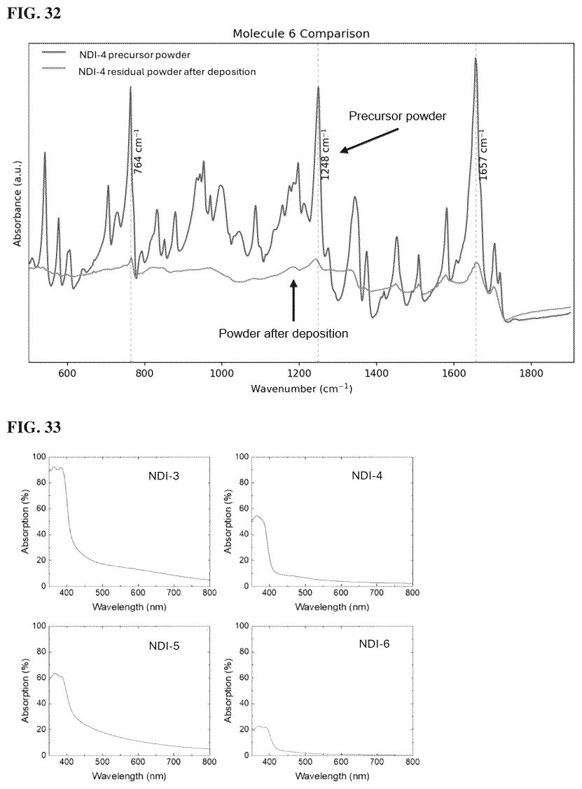

To evaluate the performance of these organic films as ETLs n-i-p solar cells with the architecture FTO/NDI-ETL/Cs_{0.09}FA_{0.9}iPbI₃/PEAI/Spiro-OMeTAD/Au were fabricated as shown schematically in FIG. 17. The efficacy of the films as ETLs for photovoltaic application was evaluated by comparing J-V curves under illumination. The two champion J-V scans for NDI-(EtPA)₂ and NDI-(PhBr)₂ are displayed in FIG. 5A. PCEs of 15.6% and 14.1% were achieved, respectively. These NDI containing devices outperform any previously reported PSCs using thermally evaporated NDI ETLs. FIG. 5B shows the distributions of the other key performance metric: V_{OC}, J_{SC}, and fill factor (FF), which display similar trends to FIG. 5B and can be found in FIG. 18A. While both NDI-based devices exhibit more variance in their performance than the reference devices, NDI-(EtPA)₂ exhibits considerably higher variance and, in general, lower efficiency than NDI-(PhBr)₂. Given the similar band gaps and frontier orbital energies of these two molecules, these differences likely arise from morphological or structural differences between the NDI films. It is hypothesized that the partial dissolution of the NDI-(EtPA)₂ film on deposition of the perovskite layer, as shown by GIWAXS and XPS, results in inconsistent film coverage and hence lower performances and higher variances in general. In Table 1 a summary of the maximum performance parameters for all synthesized molecules can be found.

TABLE 1
Key device parameters for all NDI molecules

| | | | | Max | |
|---|---|---|---|---|---|
| | Max J_{SC} | Max V_{OC} | Max | stabilized | Max PCE |
| Molecule | (mAcm-2) | (V) | FF % | MPP (%) | (%) |
| NDI - 1 | 24.08 | 0.93 | 63.48 | 5.66 | 14.1 |
| NDI - 2 | 24.55 | 0.99 | 64.99 | 6.84 | 15.4 |
| NDI - 3 | 22.27 | 0.92 | 56.27 | 2.64 | 10.0 |
| NDI - 4 | 24.74 | 0.98 | 62.83 | 13.45 | 14.1 |
| NDI - 5 | 24.42 | 0.94 | 68.02 | 12.1 | 13.5 |
| NDI - 6 | 24.62 | 0.93 | 60.14 | 12.45 | 13.7 |

To study the impact of long-term operation on the NDI transport layers a long-term device stability study was conducted. These stability measurements were conducted under 1 sun equivalent illumination at a temperature of 65° C. under a N₂ atmosphere with tracking of the stabilized PCE. This data was smoothed using a Lowess algorithm to reduce high frequency noise while accurately representing the long-term trends. Normalized and raw datasets can be seen in FIG. 5C and FIG. 19 respectively. Both devices containing NDI-(EtPA)₂ and NDI-(PhBr)₂ exhibit long-term stability over 150 hours of operation, outperforming our TiO₂ reference devices, despite the increased variability in the performance of our NDI-containing devices throughout the measurement. If it was assumed the NDI layer was removed completely on washing, an FTO/perovskite interface would be expected to be present in the device. As can be seen from FIG. 5C, this interface lacks long-term stability in its performance, with device failure occurring within 50 hours of operation. The considerable improvement in long-term performance for devices containing NDI-(EtPA)₂ is evidence of a monolayer remaining that aids in the extraction of electrons and stabilization of the perovskite/FTO interface.

### Example 7: Exemplary Preparation and Characterization of Thin Films and Devices of this Disclosure

### Substrate Cleaning

1 inch×1 inch substrates (glass, FTO, FTO patterned glass) were cleaned via sequential sonication for 15 minutes in 2% Mucasol solution, distilled water, acetone (Sigma-Aldrich, >99.5%), and finally isopropyl alcohol (Fischer Chemical). PET-ITO underwent a similar cleaning procedure with the acetone wash omitted. After cleaning, the substrates were dried using a nitrogen gun. Prior to deposition of subsequent layers substrates were placed in a UV-ozone cleaner for 15 min.

### NDI Evaporation

NDI layers were deposited via thermal evaporation using a Kurt J. Lesker MiniSpectros series low-temperature evaporator. Substrates were placed in the evaporator and the system was pumped down to at least 1×10⁻⁵ torr. The substrates were rotated at a rate of 10 rpm during the deposition procedure to ensure uniformity. Specific weights of the molecules were weighed out (≈3 mg, ≈30 mg, ≈100 mg) in a nitrogen atmosphere before being placed in the evaporator. The molecules were heated gently until deposition was recorded via a 6 MHz gold Quartz Crystal Microbalance (QCM) positioned close to the thermal source. The deposition was continued until no more rate was observed despite increasing the source temperature.

### Thin Film and Molecular Characterization

¹H Nuclear Magnetic Resonance (NMR) spectra for all molecular precursors were acquired through BrukerAvance IIIHD 500 MHz or Bruker Avance IIIHD 700 MHz instruments using either CDCl₃ or DMSO-d6 as a solvent; the residual CHCl₃peak was used as a reference for all reported chemical shifts (1H: 6=7.26 ppm, 13C: δ=77.16 ppm) and for DMSO-D6 (¹H: δ=2.50 ppm, ¹³C: δ=39.48-40 ppm).

Differential Scanning Calorimetry (DSC) was performed using a TA Instruments Q200 with heating and cooling rates of 10° C. min⁻¹. Powder samples with a mass of approximately 5 mg were used and encapsulated in closed DSC aluminum pans under a controlled N₂ atmosphere.

Thermogravimetric Analysis (TGA) was performed using a Mettler Toledo TGA2 STAR System Thermogravimetric Analyzer. 5 mg of precursor powders were heated to a temperature of 700° C. at a temperature ramp rate of 15° C. min⁻¹ in an inert atmosphere of 15 ml min⁻¹ of nitrogen.

Fourier Transform Infrared Spectroscopy (FTIR) was measured using a Nicolet 6700 FT-IR. FTIR spectra were measured via attenuated total reflectance (ATR) and to ensure close contact between the sample and the diamond, films were deposited on flexible substrates of commercially available PET-ITO. Each FTIR scan was repeated 64 times.

X-Ray Photoelectron Spectroscopy (XPS): XPS was conducted using a Thermo NEXSA G2 system using a monochromatic Al Kα X-ray source (1486.6 eV). Survey scans were acquired through averaging two measurements with a 200 eV pass energy and 1 eV energy step size. Elemental scans were acquired using 50 eV pass energy and 0.1 eV energy step size. Elemental scans were recorded for all elements expected in the NDI molecules. Elemental composition analysis was performed using the Thermo Scientific Avantage data system for surface analysis.

Ultraviolet-Photoelectron-Spectroscopy (UPS) was also conducted on a Thermo NEXSA G2 system. A He 1 source (21.22 eV) of UV light was used and a 5 V bias was placed on the system. Conductive FTO substrates were used to ensure electronic connection between the samples and the spectrometer. Onset values were calculated via extrapolation of initial electron onsets to the baseline.

Optical absorption spectra were taken with a Jasco V-630 spectrometer.

Spectroscopic ellipsometry measurements were conducted using a Woollam M-2000 ellipsometer. Transmittance measurements were used to estimate film thickness. For Silicon substrates, Cauchy and BSPline models were fitted to estimate thickness.

Contact Angle Goniometry measurements were taken using ramé-hart Goniometer/Tensiometer Model 290 and analyzed through ImageJ software via low bond axisymmetric drop shape analysis. To evaluate the surface energy, 2.5 μL of diiodomethane (nonpolar) and deionized water (polar) were deposited individually onto the surface of interest.

Grazing Incidence Wide Angle X-Ray Scattering (GIWAXS) measurements were performed at beamline 11-BM at National Synchrotron Light Source II in Brookhaven National Laboratory. The X-ray beam energy was 13.5 keV with a spot size of 0.2 mm×0.05 mm. The samples were irradiated for 10 s with an incident angle of 0.5°. Beam divergence was 1 mrad with an energy resolution of 0.7%. The data were analyzed using the SciAnalysis package provided by the beamline.

### Reference Device Fabrication

A compact layer of TiO2 (c-TiO2) was deposited via spray pyrolysis, using a solution comprising 720 μL of titanium di-isopropoxide bis(acetylacetonate) (75% in 2-propanol, Sigma-Aldrich), and 10.8 mL of 99.9% pure anhydrous ethanol (SigmaAldrich). A 3.0 L min-1 flow of oxygen was used as carrier gas to spray the solution. Spraying was conducted with a Sparmax spray gun. The prepared solution was sprayed onto substrates preheated at 450° C. 20 second spray cycles were performed with a 30 second delay between each cycle. Cycles were continued until no solution remained. The substrates were then annealed at 450° C. for another 30 minutes post deposition. Substrates were then allowed to cool to room temperature before the mesoporous TiO2 layer (TiO2-mp) was deposited via static spin-coating. A 150 mg mL-1 solution of TiO2 paste (30 nm nanoparticles, GreatSolar) in ethanol (99.9% pure, anhydrous, Sigma-Aldrich) was used. The spin-coating parameters were 2000 rpm for 10 s with an acceleration rate of 2000 rpm s⁻¹. The deposited substrates were immediately moved to a hotplate and heated at 100° C. 10 minutes. The mesoporous films were subsequently sintered by heating of the substrates at 450° C. and maintaining the temperature for 30 min. Between deposition of subsequent layers, substrates were kept in an inert nitrogen atmosphere with concentrations of O₂ and H₂O<10 ppm. To study the molecular ETLs deposited by evaporation, as described above, the TiO₂ compact and mesoporous layers were substituted with NDI thin films. A film thickness of around 30 nm was targeted.

The perovskite layer was deposited via spin coating of a 1.2 M solution of Cs_{0.09}FA_{0.91}PbI₃. Precursor powders of PbI₂ (Tokyo Chemical Industry, >98%), formamidinium iodide (FAI, Dynamo) and cesium iodide(CsI, Sigma Aldrich) were weighed inside a glovebox with a 5% stoichiometric excess of PbI₂. Precursor powders were dissolved in a 2:1 volume ratio of N,N-dimethyl formamide (DMF, Acros Organics, >99.8%):dimethyl sulfoxide (Acros Organics,>99.8%). A two-step spin-coating process was used. First, 90 μL was deposited on a static substrate before being spin-coated at 1000 rpm for 10s and then 6000 rpm for 20s. 26 seconds into the spin coating procedure, 250 μL of chlorobenzene (Sigma-Aldrich, anhydrous) was dropped onto the substrate. Substrates were then annealed at 150° C. for 10 minutes. 90 μL of phenethyl ammonium iodide (PEAI, Dynamo) solution with a concentration of 1 mgmL-1 in IPA (Sigma-Aldrich, 99.9%) was used as a surface treatment. The spin coating recipe used was 20 seconds of spinning at 5000 rpm with an acceleration rate of 5000 rpm s⁻¹. After PEAI deposition, the substrate was annealed at a temperature of 100° C. for 10 minutes.

A doped Spiro-OMeTAD solution was prepared for deposition of the Hole transport layer. 100 mg of Spiro-OMeTAD (1-Material) was dissolved in 1098.38 μL of chlorobenzene (Acros Organics, 99.9%) to form a 0.07 M solution. 18.13 μL of 1.8 M lithiumbis(trifluoromethane)sulfonimide(Li-TFSI, Sigma-Aldrich) in acetonitrile(Sigma-Aldrich, anhydrous, 99.8%), 39.45 μL of 4-tertbutylpyridine (tBP, Sigma-Aldrich, 98%) and 9.79 μL of 0.25 M tris(2-(1H-pyrazol-1-yl)-4-tert-butylpyridine)cobalt(III) tri[bis(trifluoromethane)sulfonimide](FK 209 Co (III), Sigma-Aldrich) in acetonitrile were added as dopants. 90 μL of doped Spiro-OMeTAD solution was subsequently spin coated dynamically with 3000 rpm for 30 s with an acceleration of 3000 rpm s⁻¹.

The edges of the substrates were then cleaned with DMF and acetonitrile to remove the perovskite and hole transport layers in ambient air. 50 nm of gold (KJLC, >99.999%) was deposited via thermal evaporation as a back contact. Shadow masks were used to define 8 pixels per substrate, each with an active area of 0.128 cm².

### NDI—Containing Device Fabrication

Devices were fabricated with all steps being identical to that of the reference device procedure with the exception of the ETL deposition stage. For ETL deposition NDI layers were deposited via thermal evaporation using a Kurt J. Lesker MiniSpectros series low-temperature evaporator. Substrates were placed in the evaporator and the system was pumped down to at least 1E-5 torr. The substrates were rotated at a rate 10 rpm during the deposition procedure to ensure uniformity. Specific weights of the molecules were weighed out (≈3 mg, ≈30 mg, ≈100 mg) in a nitrogen atmosphere before being placed in the evaporator. The molecules were heated gently until deposition was recorded via a Quartz Crystal Microbalance (QCM) positioned close to the thermal source.

### Thin Film Deposition

NDI thin films were drop cast onto cleaned substrates of Glass, FTO and PET-ITO. 5 mg/ml solutions of the NDI molecules were dissolved in DMSO. The solution was then dropped onto the substrates, which were subsequently heated at 100° C. for 15 minutes.

### Device Performance

The photovoltaic performance of the devices was evaluated using a Fluxim Litos Lite setup, equipped with a Wavelabs Sinus-70 AAA solar simulator with AM1.5 spectrum for excitation. The current—voltage (J-V) characteristics were acquired with forward and reverse scans ranging from −0.5 V to 1.2 V with a scan rate of 50 mV s⁻¹. The stabilized power output was acquired using an MPP tracking algorithm for 120 s. Devices were not preconditioned before measurement. Masking was used during the measurement, defining a pixel area of 0.0625 cm². Nitrogen was blown in the measurement chamber during characterization. No temperature control was applied. For the long-term thermal stability measurement, a Fluxim Litos, a stress-test platform for degradation analysis, was employed. The solar cells were stressed at temperature of 65° C. in N₂ rich environment, under 1 sun equivalent illumination without UV light and with continuous MPP tracking. During the stability measurement, automated acquisition of J-V scans in both reverse and forward directions were taken every 12 hours. Data smoothing was performed using the Lowess (locally weighted scatterplot smoothing) algorithm in OriginPro, with a window spanning the nearest 506 neighboring data points for the stability data in FIG. 5C due to some degree of variability in the measurement.

### Example 8: Exemplary Characterization Techniques Used for Thin Films and Devices of this Disclosure

### Thermogravimetric Analysis (TGA)

TGA was performed using a Mettler Toledo TGA2 STAR System Thermogravimetric Analyzer. 5 mg of precursor powders were heated to a temperature of 700C at a temperature ramp rate of 15° C./minute in an inert atmosphere of 15 ml/min of Nitrogen.

### Fourier Transform Infrared Spectroscopy (FTIR)

FTIR spectra for the thin films were measured using a Nicolet 6700 FT-IR. FTIR spectra were measure via ATR and to ensure close contact between the sample and the diamond, films were deposited on flexible substrates of commercially available PET-ITO (PROVIDER). FTIR scans were run with resolutions between 4 and 1, and each scan was repeated 64 times.

### X-Ray Photoelectron Spectroscopy (XPS)

XPS was conducted using a Thermo NEXSA G2 system using a monochromatic Al Kα X-ray source (1486.6 eV) Survey scans were acquired through averaging two measurements with a 200 eV pass energy and 1 eV energy step size. Elemental scans were acquired using 50 eV pass energy and 0.1 eV energy step size. Elemental scans were recorded for all elements expected in the NDI molecules. Elemental composition analysis was performed using the Thermo Scientific Avantage data system for surface analysis.

### Ultraviolet-Photoelectron-Spectroscopy (UPS)

UPS was also conducted on a Thermo NEXSA G2 system. A He 1 source (21.22 eV) of UV light was used and a 5V bias was placed on the system. Conductive FTO substrates were used to ensure electronic connection between the samples and the spectrometer. Onset values were calculated via extrapolation of initial electron onsets to the baseline. (REDO)

### UV-Vis Spectroscopy

UV-vis absorption spectra were taken with a Jasco V-630 spectrometer.

### Spectroscopic Ellipsometry

Ellipsometry measurements were conducted using a Woollam M-2000 elipsometer. Transmittance measurements were used to estimate film thickness. For Silicon substrates, Cauchy and BSPline models were fitted to estimate thickness.

### Contact Angle

Contact angle measurements were taken using ramé-hart Goniometer/Tensiometer Model 290 and analyzed through Image J software via low bond axisymmetric drop shape analysis. To evaluate the surface energy, 2.5 μL of diiodoylmethane (nonpolar) and deionized water (polar) were deposited individually onto the surface of interest.

### Example 9: Exemplary Synthetic Procedures for Compounds of the Disclosure

TABLE 2
Compounds of the present disclosure

| | |
|---|---|
| NDI—(PhBr)₂ (NDI-2) | 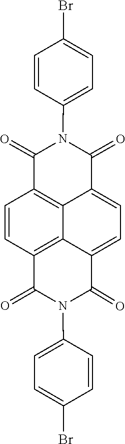 [SMILES: O=C1c2ccc3c4c(ccc(c24)C(=O)N1c1ccc(Br)cc1)C(=O)N(c1ccc(Br)cc1)C3=O] |
| NDI—(PhBr) (Hex) (NDI-3) | 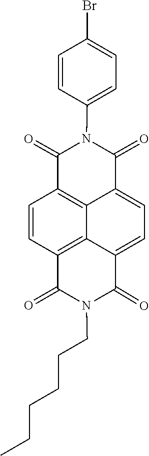 [SMILES: CCCCCCN1C(=O)c2ccc3c4c(ccc(c24)C1=O)C(=O)N(c1ccc(Br)cc1)C3=O] |
| NDI—(EtPA)₂ (NDI-1) | 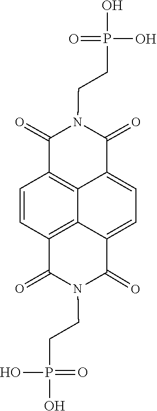 [SMILES: O=C1c2ccc3c4c(ccc(c24)C(=O)N1CCP(=O)(O)O)C(=O)N(CCP(=O)(O)O)C3=O] |
| NDI—(BzPA) (PhBr) (NDI-5) | 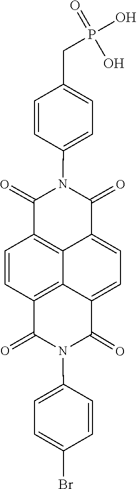 [SMILES: O=C1c2ccc3c4c(ccc(c24)C(=O)N1c1ccc(Br)cc1)C(=O)N(c1ccc(CP(=O)(O)O)cc1)C3=O] |
| NDI—(BzPA) (Hex) (NDI-4) | 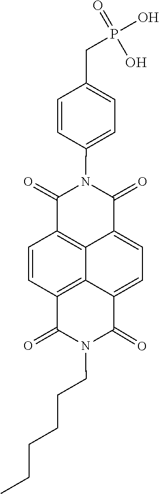 [SMILES: CCCCCCN1C(=O)c2ccc3c4c(ccc(c24)C1=O)C(=O)N(c1ccc(CP(=O)(O)O)cc1)C3=O] |

### Amine Synthesis

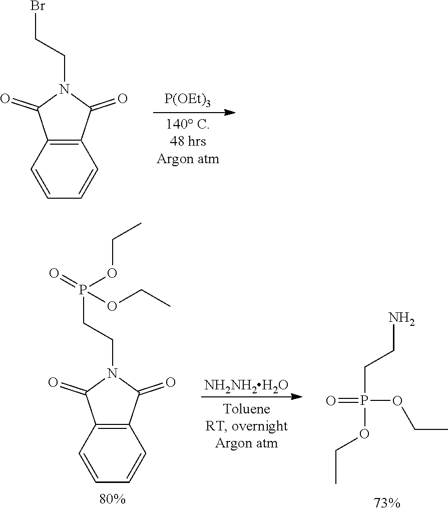

### Diethyl (2-(1,3-dioxoisoindolin-2-yl)ethyl)phosphonate

To a 100 mL round bottom flask charged with a magnetic stir bar was added N-(2-bromoethyl)phthalimide (3.6 g, 14.2 mmoles, 1 eq.) and triethyl phosphite (12 mL, 70.8 mmoles, 5 eq.) under an inert atmosphere. The vessel was then added to a hot plate at 140° C. and stirred for 48 hours. Afterwards the reaction was cooled to room temperature and excess triethyl phosphite was distilled off. The crude was purified by silica gel chromatography using 100% DCM to 100% ethyl acetate which a viscous yellow oil was obtained (3.52 g, 80% yield). ¹H NMR (500 MHz, CDCl₃) δ 7.87 (dd, J=5.5, 3.0 Hz, 2H), 7.74 (dd, J=5.5, 3.0 Hz, 2H), 4.24-4.05 (m, 4H), 4.05-3.82 (m, 2H), 2.36-2.10 (m, 2H), 1.32 (t, J=7.1 Hz, 6H). ³¹p NMR (202 MHz, CDCl₃) δ 27.39. ¹³C NMR (126 MHz, CDCl₃) δ 167.82, 134.04, 132.08, 123.32, 61.94, 61.88, 32.26, 32.24, 25.34, 24.23, 16.37, 16.32.

### Diethyl P-(2-aminoethyl)phosphonate

To a 250 mL round bottom flask charged with a magnetic stir bar was added diethyl (2-(1,3-dioxoisoindolin-2-yl)ethyl)phosphonate (3.52 g, 11.3 mmoles, 1 eq.) and 50 mL of anhydrous toluene under an inert atmosphere. The solution stirred for 10 minutes at room temperature before a solution of hydrazine monohydrate (7 mL, 135.6 mmoles, 12 eq.) was added and the reaction was left to stir overnight at room temperature. The reaction was then filtered and the precipitate was washed excessively with ethyl acetate. All of the filtrate was collected and then concentrated in vacuo to yield a pale yellow oil that was used without any further purification (1.5 g, 73% yield). ¹H NMR (500 MHz, CDCl₃) δ 4.29-3.82 (m, 4H), 2.95 (dt, J=17.1, 7.0 Hz, 2H), 1.87 (dt, J=17.9, 7.0 Hz, 2H), 1.27 (t, J=7.1 Hz, 6H). ³¹P NMR (202 MHz, CDCl₃) δ 30.86. ¹³C NMR (126 MHz, CDCl₃) δ 61.49, 61.44, 36.33, 36.29, 30.58, 29.48, 16.43, 16.38.

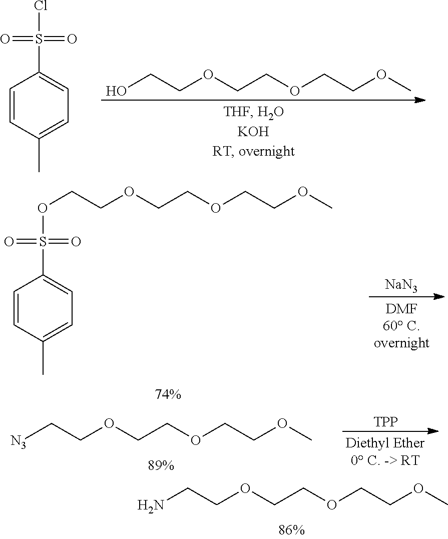

### 2-(2-(2-methoxyethoxy)ethoxy)ethyl 4-methylbenzenesulfonate

To a 250 mL round bottom flask equipped with a stir bar was added triethylene glycol monomethyl ether (13.8 g, 84 mmoles, 1 eq.) and a solution of potassium hydroxide (6.7 g, 120 mmoles, 1.4 eq.) in 25 mL of distilled water and 25 mL of tetrahydrofuran. The reaction vessel was placed on an ice bath. A solution of tosyl chloride (16.0 g, 84 mmoles, 1 eq.) in 25 mL of tetrahydrofuran was added by dropwise addition to the reaction vessel and left to stir at 0° C. for 2 hours upon completion of dropwise addition. The reaction was then left to stir overnight at room temperature. Afterwards, the reaction was concentrated under vacuum and redissolved in diethyl ether. The diethyl ether was washed with distilled water (x3) and brine. All organic layers were collected, dried over with sodium sulfate, filtered, and concentrated in vacuo to obtain a clear transparent oil (19.9 g, 74% yield). ¹H NMR (500 MHz, CDCl₃) δ 7.86-7.69 (m, 2H), 7.34 (d, J=8.1 Hz, 2H), 4.22-4.06 (m, 2H), 3.68 (dd, J=5.7, 4.0 Hz, 2H), 3.65-3.55 (m, 6H), 3.53 (dd, J=5.8, 3.5 Hz, 2H), 3.37 (s, 3H), 2.45 (s, 3H). ¹³C NMR (126 MHz, CDCl₃) δ 144.81, 132.99, 129.83, 127.97, 76.85, 71.89, 70.72, 70.55, 70.53, 69.26, 68.66, 59.02, 21.63.

### 1-azido-2-(2-(2-methoxyethoxy)ethoxy)ethane

To a 1000 mL round bottom flask charged with a stir bar was added 2-(2-(2-methoxyethoxy)ethoxy)ethyl 4-methylbenzenesulfonate (35.6 g, 111.8 mmoles, 1 eq.) and 500 mL of anhydrous dimethylformamide. Then anhydrous sodium azide (18.1 g, 278.4 mmoles, 2.5 eq.) was carefully added. The reaction stirred at 60° C. for 48 hours and then concentrated in vacuo. The crude was redissolved in diethyl ether and washed with saturated lithium chloride (x5), distilled water, and brine. All organic layers were collected, dried over with sodium sulfate, filtered, and concentrated in vacuo to obtain a clear slightly yellow oil (19.3 g, 90% yield). ¹H NMR (500 MHz, CDCl₃) δ 3.72-3.61 (m, 8H), 3.59-3.52 (m, 2H), 3.43-3.30 (m, 5H). ¹³C NMR (126 MHz, CDCl₃) δ 76.81, 71.93, 70.70, 70.66, 70.61, 70.02, 59.01, 50.69.

### 2-(2-(2-methoxyethoxy)ethoxy)ethan-1-amine

To a 500 mL round bottom flask equipped with a stir bar was added 1-azido-2-(2-(2-methoxyethoxy)ethoxy)ethane (15 g, 79.3 mmoles, 1 eq.) dissolved in 200 mL of diethyl ether. The reaction vessel was cooled to 0° C. before triphenylphosphine was added (25 g, 95.1 mmoles, 1.2 eq.). The mixture stirred for 1 hour at 0° C. and then 1 hour at room temperature. The reaction was then quenched with 80 mL of distilled water and the mixture was then vigorously stirred for 4 hours. Then 60 mL of toluene was added and left to stir overnight. The organic layer was extracted once with toluene and the aqueous layer was concentrated in vacuo to obtain a clear transparent oil (11.1 g, 86% yield). ¹H NMR (400 MHz, CDCl₃) δ 3.63 (dh, J=5.3, 2.9 Hz, 6H), 3.56-3.51 (m, 2H), 3.49 (t, J=5.2 Hz, 2H), 3.36 (s, 3H), 2.84 (t, J=5.2 Hz, 2H). ¹³C NMR (126 MHz, CDCl₃) δ 76.77, 73.38, 71.92, 70.58, 70.51, 70.25, 59.01, 41.75.

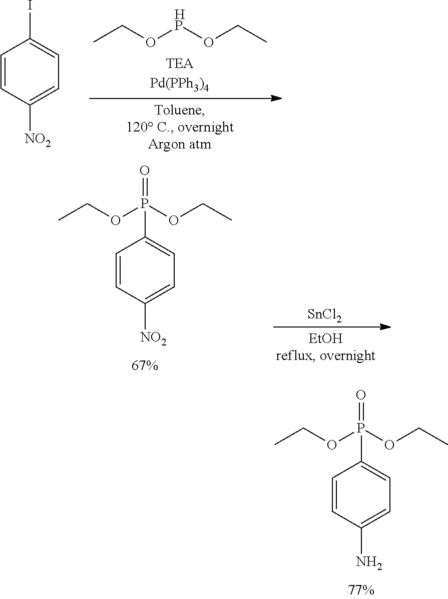

### Diethyl (4-nitrophenyl)phosphonate

To a 500 mL round bottom flask charged with a magnetic stir bar was added 1-iodo-4-nitrobenzene (10 g, 49.5 mmoles, 1 eq.), triethylamine (20 g, 198 mmoles, 4 eq.), diethyl phosphonite (7.5 g, 54.5 mmoles, 1.1 eq.), Pd(PPh₃)₄ (1.14 g, 0.99 mmoles, 0.02 eq.), and 250 mL of anhydrous toluene under inert atmosphere with a reflux condenser. The mixture was left to stir under reflux at 120° C. for 24 hours before cooling to room temperature. The reaction was filtered and the filtrate was concentrated in vacuo and then redissolved in ethyl acetate. The organic layer was washed with 1M HCl, 2 M NaOH, and then brine. All organic layers were collected, dried over with sodium sulfate, filtered, and concentrated in vacuo. The crude product was then purified via silica gel chromatography using 1:1 hexanes:EtOAC, 100% DCM, and then 100% EtOAC to obtain a dark red oil (8.1 g, 67% yield). ¹H NMR (500 MHz, CDCl₃) δ 8.39-8.14 (m, 2H), 8.14-7.88 (m, 2H), 4.32-4.00 (m, 4H), 1.33 (td, J=7.1, 0.6 Hz, 6H). ³¹P NMR (202 MHz, CDCl₃) δ 15.37. ¹³C NMR (126 MHz, CDCl₃) δ 150.25, 150.22, 136.53, 135.04, 133.00, 132.91, 132.11, 128.56, 128.46, 123.41, 123.29, 62.81, 62.77, 16.35, 16.30.

### Diethyl (4-aminophenyl)phosphonate

To a 500 mL round bottom flask equipped with a magnetic stir bar was added diethyl (4-nitrophenyl)phosphonate (4.0 g, 15.3 mmoles, 1 eq.), anhydrous tin (II) chloride (11.6 g, 61.2 mmoles, 4 eq.), and 100 mL of absolute ethanol. The mixture refluxed for 12 hours and then cooled to room temperature. The mixture was concentrated in vacuo and suspended in 2 M KOH in distilled water. A precipitate immediately formed and filtered and washed excessively with ethyl acetate. The filtrate was collected and washed with distilled water (x1) and brine (x1) and then all organics were collected dried over with sodium sulfate, filtered, and concentrated in vacuo to receive a viscous yellow oil that solidifies (2.7 g, 77% yield). ¹H NMR (500 MHz, DMSO) δ 7.33 (dd, J=12.5, 8.5 Hz, 2H), 6.74-6.50 (m, 2H), 4.05-3.79 (m, 4H), 1.20 (t, J=7.1 Hz, 6H). ³¹P NMR (202 MHz, DMSO) δ 21.82. ¹³C NMR (126 MHz, DMSO) δ 153.03, 153.00, 133.42, 133.33, 113.53, 113.41, 113.35, 111.78, 61.38, 61.34, 16.67, 16.62.

### Compound Synthesis:

### NDI-(EtPA)₂ precursor: Tetraethyl ((1,3,6,8-tetraoxo-1,3,6,8-tetrahydrobenzo[lmn][3,8]phenanthroline-2,7-diyl)bis(ethane-2,1-diyl))bis(phosphonate)

To a 250 mL round bottom flask charged with a magnetic stir bar was added napthalenetetracarboxylic dianhydride (0.73 g, 2.7 mmoles, 1 eq.) and 25 mL of glacial acetic acid. The reaction stirred for 10 minutes at 60° C. before diethyl P-(2-aminoethyl)phosphonate (1.5 g, 8.2 mmoles, 3 eq.) was added. The temperature was raised to 90° C. and the reaction was left to stir for 48 hours. Afterwards, the reaction cooled to room temperature before precipitating into cold distilled water. The precipitate was filtered and washed excessively with distilled water. The filtered powder was then placed under high vacuum to dry for 48 hours to obtain a light brown powder (1.13 g, 69.7% yield). ¹H NMR (500 MHz, CDCl₃) δ 8.79 (s, 4H), 4.59-4.42 (m, 4H), 4.30-3.93 (m, 8H), 2.44-2.16 (m, 4H), 1.37 (t, J=7.1 Hz, 12H). ³¹p NMR (202 MHz, CDCl₃) δ 27.35. ¹³C NMR (126 MHz, CDCl₃) δ 162.50, 131.11, 126.78, 126.60, 6.01, 61.96, 35.15, 24.94, 23.84, 16.47, 16.43.

### NDI-(EtPA)₂: ((1,3,6,8-tetraoxo-1,3,6,8-tetrahydrobenzo[lmn][3,8]phenanthroline-2,7-diyl)bis(ethane-2,1-diyl))bis(phosphonic acid)

To a 100 mL round bottom flask charged with a magnetic stir bar was added Tetraethyl ((1,3,6,8-tetraoxo-1,3,6,8-tetrahydrobenzo[lmn][3,8]phenanthroline-2,7-diyl)bis(ethane-2,1-diyl))bis(phosphonate) (1.13 g, 1.9 mmoles, 1 eq.) and 25 mL of anhydrous dichloromethane under an inert atmosphere. After stirring for 10 minutes at room temperature, bromotrimethylsilane (2.9 g, 19.0 mmoles, 10 eq.) was added and the reaction was left to stir overnight at room temperature. After 12 hours, 3 mL of methanol was added and left to stir for an additional 3 hours. The solution was then concentrated to complete dryness and the powder was suspended in methanol and filtered. The product was then dried under high vacuum to receive a white powder (0.74 g, 81% yield). ¹H NMR (500 MHz, DMSO) δ 8.61 (s, 4H), 4.45 (t, J=7.2 Hz, 4H), 4.25 (dt, J=11.7, 6.8 Hz, 4H), 2.13-1.81 (m, 4H). ³¹P NMR (202 MHz, DMSO) δ 22.15. ¹³C NMR (126 MHz, DMSO) δ 162.90, 159.92, 142.49, 132.14, 130.81, 128.86, 126.88, 126.61, 125.10, 115.18, 35.95, 35.10, 27.51, 26.45.

### NDI-(PhBr)_{2:} 2,7-bis(4-bromphenyl)benzo[lmn][3,8]phenanthroline-1,3,6,8(2H,7H)-tetraone

To a round bottom flask charged with a stir bar napthalenetetracarboxylic dianhydride (6.0 g, 22.4 mmol, 1 eq.) and 4-bromoaniline (9.6 g, 55.9 mmol, 2.5 eq.) were added into 100 mL of anhydrous dimethylformamide. The reaction mixture was placed on a hot plate at 120° C. and left to stir overnight. Afterwards, the reaction cooled to room temperature, was filtered, and washed with methanol to obtain a yellow powder. ¹H NMR (500 MHz, DMSO) δ 8.74 (s, 4H), 7.79 (d, J=8.5 Hz, 4H), 7.46 (d, J=8.5 Hz, 4H). ¹³C could not be obtained due to limited solubility and low resolution. ESI-MS: m/z theoretical 574.9236586, obtained 574.9240.

### Tetraethyl ((1,3,8,10-tetraoxo-1,3,8-10-tetrahydroanthra[2,1,9-def:6,5,10-d′e′f′]diiisoquinoline-2,9-diyl)bis(ethane-2,1-diyl))bis(phosphonate

To a 100 mL round bottom flask charged with a magnetic stir bar was added perylenetetracarboxylic dianhydride (1.10 g, 2.8 mmoles, 1 eq.), 1,8-diazabicyclo(5.4.0)undec-7-ene (1.64 mL, 11.0 mmoles, 4 eq.), and 20 mL of anhydrous dimethylformamide. The reaction stirred for 10 minutes at room temperature before diethyl P-(2-aminoethyl)phosphonate (2.0 g, 11.0 mmoles, 4 eq.) was added. Reaction stirred for 1 hour at room temperature before the temperature was increased to 60° C. and stirred for 24 hours. Reaction was then cooled to room temperature and then DMF was removed by roto-evaporation. The powder was then suspended in distilled water and placed in the fridge overnight. Powder was then filtered and washed with distilled water. The solid was dried under high vacuum to obtain a purple solid (1.66 g, 82.6% yield). ¹H NMR (500 MHz, CDCl₃) δ 8.65 (d, J=8.0 Hz, 4H), 8.54 (d, J=8.1 Hz, 4H), 4.50 (d, J=8.0 Hz, 4H), 4.27-4.16 (m, 8H), 2.41-2.26 (m, 4H), 1.41 (t, J=7.1 Hz, 12H). ³¹P NMR (202 MHz, CDCl₃) δ 27.92. ¹³C NMR (126 MHz, CDCl₃) δ 162.97, 134.60, 131.43, 129.30, 126.35, 123.16, 109.03, 61.98, 61.93, 34.90, 25.00, 16.51, 16.46.

### ((1,3,8,10-tetraoxo-1,3,8,10-tetrahydroanthra[2,1,9-def:6,5,10-d′e′f′]diisoquinoline-2,9-diyl)bis(ethane-2,1-diyl))bis(phosphonic acid)

To a 100 mL round bottom flask charged with a magnetic stir bar was added tetraethyl ((1,3,8,10-tetraoxo-1,3,8-10-tetrahydroanthra[2,1,9-def:6,5,10-d′e′f′]diiisoquinoline-2,9-diyl)bis(ethane-2,1-diyl))bis(phosphonate (1.46 g, 2.0 mmoles, 1 eq.) and 25 mL of anhydrous dichloromethane under an inert atmosphere. After stirring for 10 minutes at room temperature, bromotrimethylsilane (3.1 g, 20.3 mmoles, 10 eq.) was added and the reaction was left to stir overnight at room temperature. After 12 hours, 3 mL of methanol was added and left to stir for an additional 3 hours. The solution was then concentrated to complete dryness and the powder was suspended in methanol and filtered. The product was then dried under high vacuum to receive a purple powder (1.21 g, 100% yield). ¹H NMR (500 MHz, DMSO) δ 8.63 (s, 4H), 8.36 (s, 4H), 4.26 (s, 4H), 2.07-2.00 (m, 4H). ³¹P NMR (202 MHz, DMSO) δ 22.49. No ¹³C NMR could be obtained due to low solubility.

### NDI-(PhBr)-(Hex) Precursor: 7-hexyl-1 H-isochromeno[6,5,4-def]isoquinoline-1,3,6,8(7 H)-tetraone

To a 1000 mL round bottom flask equipped with a magnetic stir bar was added napthalenetetracarboxylic dianhydride (10 g, 37.3 mmoles, 1 eq.) suspended in distilled water and 170 mL of a 1 M KOH solution was added. The solution was left to stir at 60° C. for 30 minutes until all starting materials were fully dissolved. Afterwards, the pH of the solution was adjusted to around 5-6 using a solution of 1 M phosphoric acid. Then hexylamine (4.90 mL, 37.3 mmoles, 1 eq.) was added in one portion and the pH of the solution was again adjusted to 5-6 using a 1 M solution of phosphoric acid. The reaction was then heated to reflux for 24 hours and then cooled to room temperature. The solution was filtered and then the filtrate was acidified using about 10-20 mL of glacial acetic acid. A white precipitate immediately formed and the solution was filtered. The precipitate was washed excessively with distilled water. Afterwards, the filtered precipitate was dried in a high vacuum oven overnight where a white powder was received and used without any further purifications (11.4 g, 88% yield). ¹H NMR (500 MHz, DMSO) δ 8.52 (d, J=7.5 Hz, 2H), 8.07 (d, J=7.6 Hz, 2H), 4.04 (dd, J=8.4, 6.7 Hz, 2H), 1.65 (ddd, J=9.1, 6.2, 2.0 Hz, 2H), 1.37-1.26 (m, 6H), 0.94-0.81 (m, 3H). ¹³C NMR (126 MHz, CDCl₃) δ 170.05, 163.51, 130.60, 128.96, 128.46, 125.98, 123.80, 31.43, 27.84, 26.64, 22.44, 14.39.

### NDI-(PhBr)-(Hex): 2-(4-bromophenyl)-7-hexylbenzo[lmn][3,8]phenanthroline-1,3,6,8(2H, 7H)-tetraone

To a 250 mL round bottom flask charged with a magnetic stir bar was added 7-hexyl-1 H-isochromeno[6,5,4-def]isoquinoline-1,3,6,8(7 H)-tetraone (2.3 g, 6.3 mmoles, 1 eq.) and 100 mL of glacial acetic acid. The solution was heated to 60° C. and stirred for about 10 minutes before 4-bromoaniline (2.3 g, 13.3 mmoles, 1.5 eq) was added. The solution was further heated to 110° C. and left to stir overnight. Afterwards, the solution was then cooled to room temperature, precipitated into distilled water, and filtered. The precipitate was dried in under high vacuum and then purified by silica gel chromatography using a gradient of 100% DCM, 1:1 DCM: EtOAC mixture, and 100% EtOAC. A pure white powder was obtained (2.60 g, 82% yield). ¹H NMR (500 MHz, CDCl₃) δ 8.83 (s, 4H), 7.79-7.67 (m, 2H), 7.26-7.19 (m, 2H), 4.33-4.09 (m, 2H), 1.78 (t, J=7.7 Hz, 2H), 1.47 (d, J=7.8 Hz, 2H), 1.43-1.30 (m, 4H), 0.97-0.87 (m, 3H). ¹³C NMR (126 MHz, CDCl₃) δ 162.85, 162.71, 133.55, 132.84, 131.151, 131.05, 130.25, 127.14, 127.07, 126.86, 126.45, 123.33, 41.11, 31.50, 28.04, 26.76, 22.56, 14.06.

### NDI-(EtPA)-(Hex) precursor: Diethyl (4-(7-hexyl-1,3,6,8-tetraoxo-3,6,7,8-tetrahydrobenzo[lmn][3,8]phenanthroline-2(1 H)-yl)benzyl)phosphonate

To a 250 mL round bottom flask charged with a magnetic stir bar was added 7-hexyl-1 H-isochromeno[6,5,4-def]isoquinoline-1,3,6,8(7 H)-tetraone (2.0 g, 5.7 mmoles, 1 eq.) and 100 mL of glacial acetic acid. The solution was heated to 60° C. and stirred for about 10 minutes before diethyl (4-aminobenzyl)phosphonate (2.8 g, 11.4 mmoles, 1.5 eq) was added. The solution was further heated to 110° C. and left to stir overnight. Afterwards, the solution was then cooled to room temperature, precipitated into distilled water, and filtered. The precipitate was dried in under high vacuum and then purified by silica gel chromatography using a gradient of 100% DCM, 1:1 DCM: EtOAC mixture, and 100% EtOAC. A pure bright yellow powder was obtained (2.10 g, 64% yield). ¹H NMR (500 MHz, CDCl₃) δ 8.82 (s, 4H), 7.54 (dd, J=8.5, 2.5 Hz, 2H), 7.32-7.29 (m, 2H), 4.28-4.20 (m, 2H), 4.10 (ddd, J=8.5, 7.0, 1.4 Hz, 4H), 3.37-3.07 (m, 2H), 1.78 (d, J=7.7 Hz, 2H), 1.53-1.44 (m, 2H), 1.38 (dt, J=6.5, 3.2 Hz, 4H), 1.31 (t, J=7.1 Hz, 6H), 0.93 (t, J=7.0 Hz, 3H). ³¹P NMR (202 MHz, CDCl₃) δ 26.23. ¹³C NMR (126 MHz, CDCl₃) δ 162.97, 162.77, 133.37, 132.95, 131.35, 131.03, 130.95, 130.90, 128.65, 128.62, 127.09, 127.01, 126.87, 126.70, 62.36, 62.31, 41.08, 34.23, 33.13, 351.50, 28.04, 26.75, 22.55, 16.45, 16.40, 14.04.

### NDI-(PhBr)-(Hex): (4-(7-hexyl-1,3,6,8-tetraoxo-3,6,7,8-tetrahydrobenzo[lmn][3,8]phenanthroline-2(1 H)-yl)benzyl)phosphonic acid

To a 100 mL round bottom flask charged with a magnetic stir bar was added diethyl (4-(7-hexyl-1,3,6,8-tetraoxo-3,6,7,8-tetrahydrobenzo[lmn][3,8]phenanthroline-2(1 H)-yl)benzyl)phosphonate (2.0 g, 3.4 mmoles, 1 eq.) and 25 mL of anhydrous dichloromethane under an inert atmosphere. After stirring for 10 minutes at room temperature, bromotrimethylsilane (5.2 g, 34.1 mmoles, 10 eq.) was added and the reaction was left to stir overnight at room temperature. After 12 hours, 3 mL of methanol was added and left to stir for an additional 3 hours. The solution was then concentrated to complete dryness and the powder was suspended in methanol and filtered. The product was then dried under high vacuum to receive a white powder (1.77 g, 100% yield). ¹H NMR (500 MHz, DMSO) δ 8.82-8.49 (m, 4H), 7.42 (dd, J=8.3, 2.4 Hz, 2H), 7.34 (d, J=8.2 Hz, 2H), 4.09-4.05 (m, 2H), 3.12-3.03 (m, 2H), 1.68 (dd, J=8.8, 6.4 Hz, 2H), 1.43-1.36 (m, 2H), 1.33 (tt, J=5.9, 2.9 Hz, 4H), 0.88 (s, 3H). ³¹P NMR (202 MHz, DMSO) δ 21.25. ¹³C NMR (126 MHz, DMSO) δ 163.41, 1631.11, 135.18, 135.11, 133.90, 130.92, 130.90, 130.78, 130.73, 128.96, 127.35, 127.02, 126.82, 126.69, 36.20, 35.15, 31.42, 27.81, 26.65, 22.44, 14.38.

### NDI-(EtPA)-(PhBr) precursor: 7-(4-bromophenyl)-1H-isochromeno[6,5,4-def]isoquinoline-1,3,6,8(7 H)-tetraone

To a 1000 mL round bottom flask equipped with a magnetic stir bar was added napthalenetetracarboxylic dianhydride (10 g, 37.3 mmoles, 1 eq.) suspended in distilled water and 170 mL of a 1 M KOH solution. The solution was left to stir at 60° C. for 30 minutes until all starting materials were fully dissolved. Afterwards, the pH of the solution was adjusted to around 5-6 using a solution of 1 M phosphoric acid. Then 4-bromoaniline (6.4 g, 37.3 mmoles, 1 eq.) was added in one portion and the pH of the solution was again adjusted to 5-6 using a 1 M solution of phosphoric acid. The reaction was then heated to reflux for 24 hours and then cooled to room temperature. The solution was filtered and then the filtrate was acidified using about 10-20 mL of glacial acetic acid. A white precipitate immediately formed and the solution was filtered. The precipitate was washed excessively with distilled water. Afterwards, the filtered precipitate was dried in a high vacuum oven overnight where a white powder was received and used without any further purifications (8.7 g, 55% yield).

### NDI-(EtPA)-(PhBr) precursor: Diethyl (4-(7-(4-bromophenyl)-1,3,6,8-tetraoxo-3,6,7,8-tetrahydrobenzo[lmn][3,8]phenanthroline-2(1 H)-yl)benzyl)phosphonate

To a 250 mL round bottom flask charged with a magnetic stir bar was added 7-(4-bromophenyl)-1H-isochromeno[6,5,4-def]isoquinoline-1,3,6,8(7 H)-tetraone (2.0 g, 4.7 mmoles, 1 eq.) and 100 mL of glacial acetic acid. The solution was heated to 60° C. and stirred for about 10 minutes before diethyl (4-aminobenzyl)phosphonate (2.3 g, 9.47 mmoles, 1.5 eq) was added. The solution was further heated to 110° C. and left to stir overnight. Afterwards, the solution was then cooled to room temperature, precipitated into distilled water, and filtered. The precipitate was dried in under high vacuum and then purified by silica gel chromatography using a gradient of 100% DCM, 1:1 DCM: EtOAC mixture, and 1:3 MeOH:DCM. A yellow powder was obtained (1.5 g, 50% yield). ¹H NMR (500 MHz, CDCl₃) δ 8.87 (s, 4H), 7.74 (d, J=8.2 Hz, 2H), 7.56 (dd, J=8.3, 2.3 Hz, 2H), 7.32 (d, J=7.9 Hz, 2H), 7.26 (d, J=8.2 Hz, 2H), 4.12 (q, J=7.3 Hz, 4H), 3.40-3.15 (m, 2H), 1.32 (t, J=7.0 Hz, 6H). ³¹P NMR (202 MHz, CDCl₃) δ 26.22. ¹³C NMR (126 MHz, CDCl₃) δ 162.83, 162.76, 132.86, 131.60, 131.46, 131.00, 130.24, 128.62, 127.18, 126.81, 62.38, 30.93, 16.45, 16.40.

### NDI-(EtPA)-(PhBr): Diethyl (4-(7-(4-bromophenyl)-1,3,6,8-tetraoxo-3,6,7,8-tetrahydrobenzo[lmn][3,8]phenanthroline-2(1 H)-yl)benzyl)phosphonic acid

To a 100 mL round bottom flask charged with a magnetic stir bar was added diethyl (4-(7-(4-bromophenyl)-1,3,6,8-tetraoxo-3,6,7,8-tetrahydrobenzo[lmn][3,8]phenanthroline-2(1 H)-yl)benzyl)phosphonate (1.4 g, 2.2 mmoles, 1 eq.) and 25 mL of anhydrous dichloromethane under an inert atmosphere. After stirring for 10 minutes at room temperature, bromotrimethylsilane (3.3 g, 21.6 mmoles, 10 eq.) was added and the reaction was left to stir overnight at room temperature. After 12 hours, 3 mL of methanol was added and left to stir for an additional 3 hours. The solution was then concentrated to complete dryness and the powder was suspended in methanol and filtered. The product was then dried under high vacuum to receive a yellow powder (1.10 g, 100% yield). ¹H NMR (500 MHz, DMSO) δ 8.73 (s, 4H), 7.78 (d, J=8.4 Hz, 2H), 7.48-7.40 (m, 4H), 7.36 (d, J=8.0 Hz, 2H), 3.06 (s, 2H). ³¹P NMR (202 MHz, DMSO) δ 21.22. ¹³C NMR (126 MHz, DMSO) δ 163.47, 163.34, 135.48, 133.92, 132.51, 131.83, 130.94, 130.79, 130.74, 128.99, 127.55, 127.38, 127.17, 122.21.

### 7-(2-(2-(2-methoxyethoxy)ethoxy)ethyl)-1 H-isochromeno[6,5,4-def]isoquinoline-1,3,6,8(7 H)-tetraone

To a 1000 mL round bottom flask equipped with a magnetic stir bar was added napthalenetetracarboxylic dianhydride (10 g, 37.3 mmoles, 1 eq.) suspended in distilled water and 170 mL of a 1 M KOH solution. The solution was left to stir at 60° C. for 30 minutes until all starting materials were fully dissolved. Afterwards, the pH of the solution was adjusted to around 5-6 using a solution of 1 M phosphoric acid. Then X (6.1 g, 37.3 mmoles, 1 eq.) was added in one portion and the pH of the solution was again adjusted to 5-6 using a 1 M solution of phosphoric acid. The reaction was then heated to reflux for 24 hours and then cooled to room temperature. The solution was filtered and then the filtrate was acidified using about 10-20 mL of glacial acetic acid. The solution was then rotovaped to complete dryness and then about 100 mL of distilled water was used to precipitate a white powder. The solution was filtered and the precipitate was dried under high vacuum. A white powder was received and used without any further purifications (12 g, 80% yield). ¹H NMR (500 MHz, D₂O) δ 8.42 (d, J=6.5 Hz, 2H), 7.94 (d, J=6.5 Hz, 3H), 4.23 (s, 2H), 3.76 (t, J=5.7 Hz, 2H), 3.60 (dd, J=5.7, 2.9 Hz, 2H), 3.55-3.44 (m, 2H), 3.38 (dd, J=5.9, 3.1 Hz, 2H), 3.21 (dd, J=5.3, 3.3 Hz, 2H), 3.09 (s, 3H). ¹³C NMR (126 MHz, D₂O) δ 173.93, 1372.32, 165.20, 141.21, 136.02, 133.30, 131.22, 128.66, 128.06, 127.91, 127.81, 125.01, 122.82, 70.83, 69.68, 69.46, 69.39, 67.41, 57.8, 39.41.

### Diethyl (4-(7-(2-(2-(2-methoxyethoxy)ethoxy)ethyl)-1,3,6,8-tetraoxo-3,6,7,8-tetrahyddrobenzo[lmn][3,8]phenanthroline-2(1 H)-yl)benzyl)phosphonate

To a 250 mL round bottom flask charged with a magnetic stir bar was added 7-(2-(2-(2-methoxyethoxy)ethoxy)ethyl)-1 H-isochromeno[6,5,4-def]isoquinoline-1,3,6,8(7 H)-tetraone (2.0 g, 4.8 mmoles, 1 eq.) and 100 mL of glacial acetic acid. The solution was heated to 60° C. and stirred for about 10 minutes before diethyl (4-aminobenzyl)phosphonate (1.8 g, 7.3 mmoles, 1.5 eq) was added. The solution was further heated to 110° C. and left to stir overnight. Afterwards, the solution was then cooled to room temperature, precipitated into distilled water, and filtered. The precipitate was dried in under high vacuum and then purified by silica gel chromatography using a gradient of 1:3 DCM: Hexanes, 100% DCM, and 1:1 DCM: EtOAC mixture. A yellow fluffy powder was obtained (0.89 g, 35% yield). ¹H NMR (500 MHz, CDCl₃) δ 8.81 (s, 4H), 7.56-7.52 (m, 2H), 7.30 (d, J=7.9 Hz, 2H), 4.50 (t, J=5.9 Hz, 2H), 4.12-4.09 (m, 4H), 3.89 (t, J=5.9 Hz, 2H), 3.74 (dd, J=5.8, 3.8 Hz, 2H), 3.68-3.57 (m, 4H), 3.52-3.46 (m, 2H), 3.35 (s, 3H), 3.31-3.25 (m, 2H), 1.33-1.30 (m, 6H). ³¹p NMR (202 MHz, CDCl₃) δ 26.25. ¹³C NMR (126 MHz, CDCl₃) δ 162.94, 162.84, 133.35, 133.01, 131.44, 131.33, 131.08, 130.96, 130.9, 128.65, 128.62, 127.07, 126.93, 126.75, 39.67, 71.89, 70.66, 70.53, 70.15, 67.82, 62.38, 62.32, 59.01, 34.23, 33.13, 16.44, 16.40.

### (4-(7-(2-(2-(2-methoxyethoxy)ethoxy)ethyl)-1,3,6,8-tetraoxo-3,6,7,8-tetrahyddrobenzo[lmn][3,8]phenanthroline-2(1 H)-yl)benzyl)phosphonic acid

To a 100 mL round bottom flask charged with a magnetic stir bar was added diethyl (4-(7-(2-(2-(2-methoxyethoxy)ethoxy)ethyl)-1,3,6,8-tetraoxo-3,6,7,8-tetrahyddrobenzo[lmn][3,8]phenanthroline-2(1 H)-yl)benzyl)phosphonate (2.9 g, 4.5 mmoles, 1 eq.) and 25 mL of anhydrous dichloromethane under an inert atmosphere. After stirring for 10 minutes at room temperature, bromotrimethylsilane (7 g, 45.4 mmoles, 10 eq.) was added and the reaction was left to stir overnight at room temperature. After 12 hours, 3 mL of methanol was added and left to stir for an additional 3 hours. The solution was then concentrated to complete dryness and the powder was suspended in methanol and filtered. The product was then dried under high vacuum to receive a light yellow powder (1.8 g, 72% yield). ¹H NMR (500 MHz, DMSO) δ 8.70 (q, J=7.6 Hz, 4H), 7.42 (dd, J=8.4, 2.4 Hz, 2H), 7.33 (d, J=8.0 Hz, 2H), 4.29 (t, J=6.4 Hz, 2H), 3.72 (t, J=6.4 Hz, 2H), 3.58 (dd, J=5.7, 3.9 Hz, 4H), 3.34 (t, J=3.2 Hz, 4H), 3.18 (s, 3H), 3.05 (s, 2H). ³¹P NMR (202 MHz, DMSO) δ 20.92. ¹³C NMR (126 MHz, DMSO) δ 163.42, 163.18, 131.02, 130.91, 130.76, 128.91, 127.47, 127.07, 126.71, 72.78, 71.67, 70.15, 70.05, 67.30, 60.61, 58.48.

### Tetraethyl ((1,3,6,8-tetraoxo-1,3,6,8-tetrahydrobenzo[lmn][3,8]phenanthroline-2,-diyl)bis(4,1-phenylene))bis(phosphonate)

To a 250 mL round bottom flask charged with a magnetic stir bar was added napthalenetetracarboxylic dianhydride (0.78 g, 2.9 mmoles, 1 eq.) and 25 mL of glacial acetic acid. The reaction stirred for 10 minutes at 60° C. before diethyl P-(2-aminoethyl)phosphonate (2.0 g, 8.7 mmoles, 3 eq.) was added. The temperature was raised to 90° C. and the reaction was left to stir for 48 hours. Afterwards, the reaction cooled to room temperature before precipitating into cold distilled water. The precipitate was filtered and washed excessively with distilled water. The filtered powder was then placed under high vacuum to dry for 24 hours and then purified via silica gel chromatography using 100% DCM to 20% MeOH in DCM to obtain a light brown powder (1.13 g, 50% yield). ¹H NMR (500 MHz, CDCl₃) δ 8.89 (s, 4H), 8.07 (dd, J=13.0, 8.4 Hz, 4H), 7.57-7.41 (m, 4H), 4.38-4.09 (m, 8H), 1.41 (t, J=7.1 Hz, 12H). ³¹P NMR (202 MHz, CDCl₃) δ 17.67. ¹³C NMR (126 MHz, CDCl₃) δ 162.63, 153.89, 138.14, 133.19, 133.10, 131.66, 128.91, 128.78, 126.96, 62.47, 62.43, 32.44, 16.43, 16.38.

### ((1,3,6,8-tetraoxo-1,3,6,8-tetrahydrobenzo[lmn][3,8]phenanthroline-2,-diyl)bis(4,1-phenylene))bis(phosphonic acid)

To a 100 mL round bottom flask charged with a magnetic stir bar was added tetraethyl ((1,3,6,8-tetraoxo-1,3,6,8-tetrahydrobenzo[lmn][3,8]phenanthroline-2,-diyl)bis(4,1-phenylene))bis(phosphonate)(0.8 g, 1.2 mmoles, 1 eq.) and 25 mL of anhydrous dichloromethane under an inert atmosphere. After stirring for 10 minutes at room temperature, bromotrimethylsilane (1.8 g, 11.6 mmoles, 10 eq.) was added and the reaction was left to stir overnight at room temperature. After 12 hours, 3 mL of methanol was added and left to stir for an additional 3 hours. The solution was then concentrated to complete dryness and the powder was suspended in methanol and filtered. The product was then dried under high vacuum to receive a light tan/brown powder (0.7 g, 100% yield). ¹H NMR (500 MHz, DMSO) δ 8.74 (s, 4H), 7.97-7.77 (m, 4H), 7.68-7.47 (m, 4H), 3.17 (s, 4H). ³¹P NMR (202 MHz, DMSO) δ 12.30. ¹³C NMR (126 MHz, DMSO) δ 163.39, 138.39, 131.82, 131.73, 130.95, 129.45, 139.33, 127.49, 127.22, 107.35, 49.07.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound of Formula (I) or Formula (II): 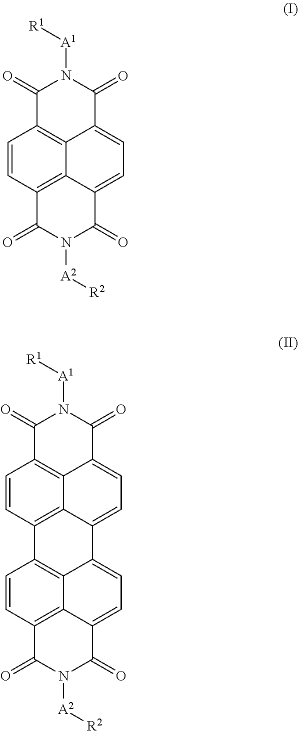
wherein:
A¹ is alkylene or arylene;
A² is selected from the group consisting of alkylene, alkoxylene, or arylene;
R¹ is selected from the group consisting of phosphate, phosphonate, -alkylene-phosphate, -alkylene-phosphonate, and halo; and
R² is selected from the group consisting of phosphate, phosphonate, -alkylene-phosphate, -alkylene-phosphonate, halo, and alkyl.

2. (canceled)

3. The compound of claim 1, wherein A¹ is methylene or ethylene.

4.-5. (canceled)

6. The compound of claim 1, wherein A¹ is phenylene.

7. (canceled)

8. The compound of claim 1, wherein A² is methylene or ethylene.

9.-12. (canceled)

13. The compound of claim 1, wherein A² is phenylene.

14. The compound of claim 1, wherein R¹ is selected from the group consisting of phosphate, phosphonate, -alkylene-phosphate, and -alkylene-phosphonate.

15.-17. (canceled)

18. The compound of claim 14, wherein R¹ is -alkylene-phosphonate, and the -alkylene-phosphonate is -methylene-phosphonate or -ethylene-phosphonate.

19.-20. (canceled)

21. The compound of claim 1, wherein R¹ is bromo.

22. The compound of claim 1, wherein R² is selected from the group consisting of phosphate, phosphonate, -alkylene-phosphate, and -alkylene-phosphonate.

23.-25. (canceled)

26. The compound of claim 22, wherein R² is -alkylene-phosphonate, and the -alkylene-phosphonate is -methylene-phosphonate or -ethylene-phosphonate.

27.-28. (canceled)

29. The compound of claim 1, wherein R² is bromo.

30. (canceled)

31. The compound of claim 1, wherein R² is hexyl.

32. The compound of claim 1, wherein the compound is selected from the group consisting of:

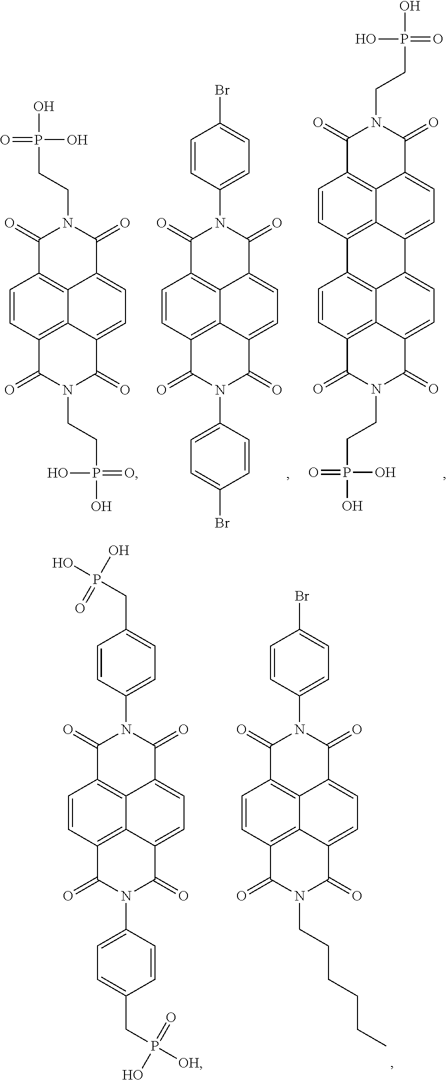

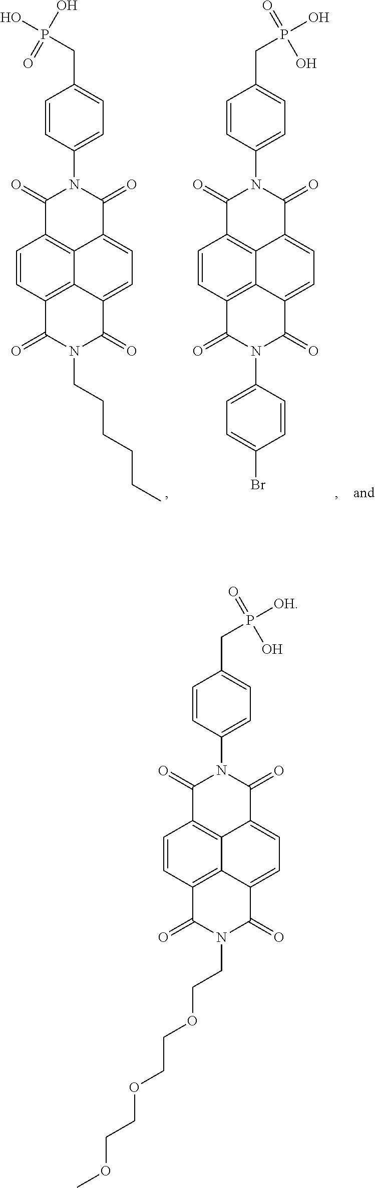 [SMILES: CCCCCCN1C(=O)c2ccc3c4c(ccc(c24)C1=O)C(=O)N(c1ccc(CP(=O)(O)O)cc1)C3=O ; O=C1c2ccc3c4c(ccc(c24)C(=O)N1c1ccc(Br)cc1)C(=O)N(c1ccc(CP(=O)(O)O)cc1)C3=O ; COCCOCCOCCN1C(=O)c2ccc3c4c(ccc(c24)C1=O)C(=O)N(c1ccc(CP(=O)(O)O)cc1)C3=O]

33. A thin film comprising a compound of claim 1.

34.-62. (canceled)

63. A method of making a thin film, the method comprising:
combining a compound of claim 1 with a solvent, thereby forming a first mixture;
contacting a substrate with the first mixture, thereby forming a treated substrate; and
heating the treated substrate, thereby forming the thin film.

64.-81. (canceled)

82. A photovoltaic device comprising:
a substrate;
an electron transport layer comprising a compound of claim 1;
an active layer;
an interface layer;
a hole transport layer; and
a metal layer.

83.-138. (canceled)

139. A method of making a photovoltaic device, the method comprising:
contacting a substrate with a first solution comprising a compound of claim 1, thereby forming an electron transport layer disposed along at least one surface of the substrate;
contacting the electron transport layer with a second solution comprising an active layer precursor, thereby forming an active layer disposed along at least one surface of the electron transport layer;
contacting the active layer with a third solution comprising an interface compound, thereby forming an interface layer disposed along at least one surface of the electron transport layer;
contacting the interface layer with a fourth solution comprising a hole transport compound, thereby forming a hole transport layer disposed along at least one surface of the interface layer;
contacting the hole transport layer with a metal, thereby forming a metal layer disposed along at least one surface of the hole transport layer;
thereby forming the photovoltaic device.

140.-180. (canceled)

181. An organic light-emitting diode (OLED) comprising a thin film of claim 33.

182. A photodetector comprising a thin film of claim 33.

183. An organic field-effect transistor (OFET) comprising a compound of claim 1.
